(19) United States
(12) Patent Application Publication
(10) Pub. No.: US 2025/0329468 A1
(43) Pub. Date: Oct. 23, 2025

(54) SYSTEMS, DEVICES, AND METHODS FOR GENERATING MACHINE LEARNING MODELS AND USING THE MACHINE LEARNING MODELS FOR EARLY PREDICTION AND PREVENTION OF PREECLAMPSIA

(71) Applicants: NX PRENATAL INC., Louisville, KY (US); The Brigham and Women’s Hospital, Inc., Boston, MA (US)

(72) Inventors: Kevin P. ROSENBLATT, Bellaire, TX (US); Thomas F. McELRATH, Dover, MA (US); Brian D. BROHMAN, Louisville, KY (US); Robert C. DOSS, Lexington, KY (US)

(21) Appl. No.: 19/256,018

(22) Filed: Jun. 30, 2025

Related U.S. Application Data
(63) Continuation of application No. 16/945,642, filed on Jul. 31, 2020, now abandoned.
Application No. 16/945,642 is a continuation of application No. PCT/US2019/016188, filed on Jan. 31, 2019.
(60) Provisional application No. 62/624,626, filed on Jan. 31, 2018.
(60) Provisional application No. 62/641,135, filed on Mar. 9, 2018.

(51) Int. Cl. G16H 50/30; G16B 5/20; G16B 40/00; G16H 10/40; G16H 50/20; G16H 50/70

(52) U.S. Cl. CPC G16H 50/30; G16B 5/20; G16B 40/00; G16H 10/40; G16H 50/20; G16H 50/70

(57) ABSTRACT

Disclosed herein are methods and systems for determining risk of preeclampsia. The system can include (a) a computer comprising: (i) a processor; and (II) a memory, coupled to the processor, the memory storing a module comprising: (1) test data for a sample from a subject including values indicating a quantitative measure of one or more markers; (2) a classification rule which, based on values including the measurements, classifies the subject as being at risk of preeclampsia, wherein the classification rule is configured to have a sensitivity of at least 75%, at least 85% or at least 95%; and (3) computer executable instructions for implementing the classification rule on the test data.

### CROSS-REFERENCE TO RELATED APPLICATIONS

This application is a continuation of U.S. patent application Ser. No. 16/945,642, filed Jul. 31, 2020, which is a continuation of International Patent Application No. PCT/US2019/016188, filed Jan. 31, 2019, which claims the benefit under 35 U.S.C. § 119(e) of U.S. Provisional Patent Application No. 62/624,626, filed Jan. 31, 2018 and 62/641,135, filed Mar. 9, 2018. The contents of these applications are incorporated herein by reference in their entireties.

### BACKGROUND

Preeclampsia (PE) is a condition of pregnant women and is characterized by hypertension (high blood pressure) and proteinuria (protein in the urine), which can lead to eclampsia or convulsions. Preeclampsia generally develops during middle to late pregnancy and up to 6 weeks after delivery, though it can sometimes appear earlier than 20 weeks or in the first trimester. It typically occurs in first pregnancies, and women who have had PE are more likely to have the same condition in the subsequent pregnancies.

PE is estimated to affect 8,370,000 women worldwide every year and is a major cause of maternal, fetal, and neonatal morbidity and mortality. PE is responsible for approximately 7%-9% of neonatal morbidity and mortality. In the U.S., it is reported to affect 200,000 pregnant women and is estimated to cause approximately $10 billion in healthcare costs. A majority of the costs (about 80%) are associated with early-onset PE (e.g., PE that develops before 35 weeks gestation) In developing countries, preeclampsia accounts for around 40-60% of maternal deaths.

Preeclampsia sometimes develops without any symptoms. High blood pressure may develop slowly or suddenly in women whose blood pressure had been normal. Other symptoms can include sudden swelling, mostly in the face and hand, sudden weight gain, headache, and change in vision, sometimes seeing flashing lights, malaise, shortness of breath, vomiting, decrease in urine output, and decrease in platelets in blood. Some women may develop complications of PE, these symptoms include fetal growth restriction, preterm delivery (PTD), placental abruption, HELLP syndrome, eclampsia, other organ damage (e.g., liver and kidney), and cardiovascular disease. Some women may also develop other complications such as intrauterine growth restriction (IUGR) and pregnancy induced hypertension (PIH).

PE can strike quickly, sometimes without any symptoms, potentially causing severe and immediate complications such as eclampsia, seizures and organ failure that threaten the health of the fetus and mother unless delivery is induced or produced surgically.

The cause of PE is unclear. Generally, women who have obesity, diabetes, lupus, immune disorders, carrying more than one fetus and pre-pregnancy high blood pressure, or kidney disease may have higher risk for preeclampsia. Other risk factors can include age, and new paternity. Women whose mother or sister had PE also have a higher risk for it.

PE can lead to long term health impacts on the mother and baby. Women who had PE may have an increased risk of hypertension and maternal coronary disease later in life. Women who had PE that leads to preterm delivery may be more prone to death from cardiovascular disease compared with women who do not develop PE and whose pregnancy goes to term. Babies who are born with reduced fetal growth or preterm delivery are more prone to have cardiovascular disease, hypertension diabetes, or mental or neurodevelopmental disorders (e.g., attention deficit disorder) later in life. Some children with developmental disorders such as autism spectrum disorder are reported being more than twice likely to be born to mothers with PE during the pregnancy.

Currently, diagnosis of PE requires both positive findings of hypertension and proteinuria.

Possible treatments for PE may include medications to lower blood pressure, corticosteroids, anticonvulsant medications, hospitalization, and, ultimately, delivery.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows a schematic and statistical workflow for identification of proteins associated with preeclampsia, related to Examples 2 and 3, and related to FIGS. 3, 4A, 4B and 5.

FIG. 2 shows biological functions with which biomarkers for increased risk of preeclampsia are associated. This represents biomarkers identified application of the statistical workflow in FIG. 1.

FIG. 3 shows 29 panels of biomarkers for preeclampsia from internal model generation before curation against the STRING protein database.

FIG. 4A and FIG. 4B show 56 panels of biomarkers for preeclampsia from model generation on a test set of samples before curation against the STRING protein database.

FIG. 5 shows 24 panels of protein biomarkers for preeclampsia after curation against the STRING protein database.

### SUMMARY

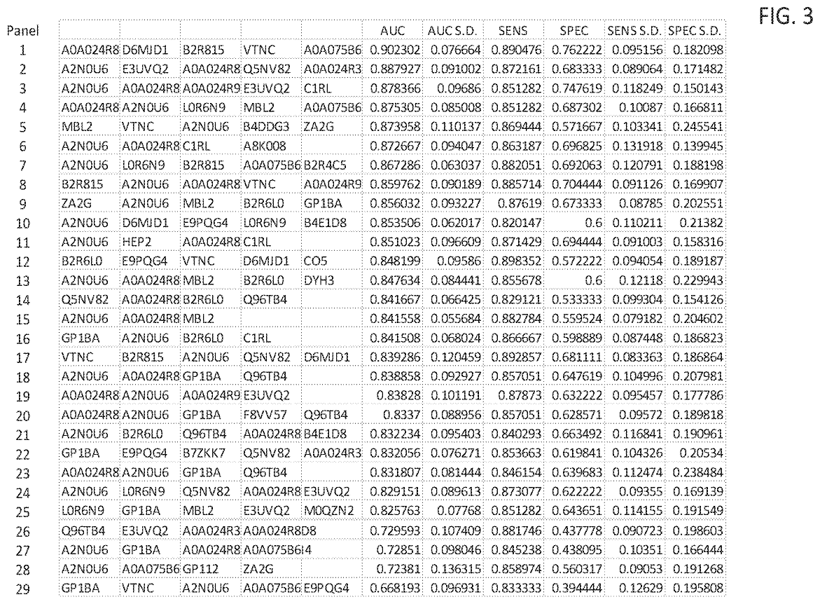

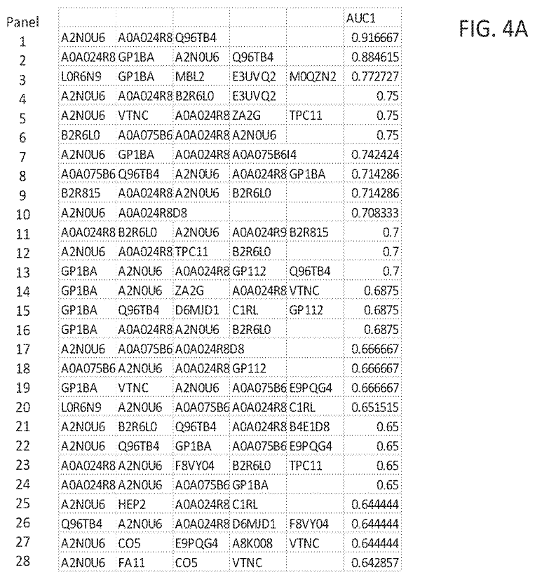

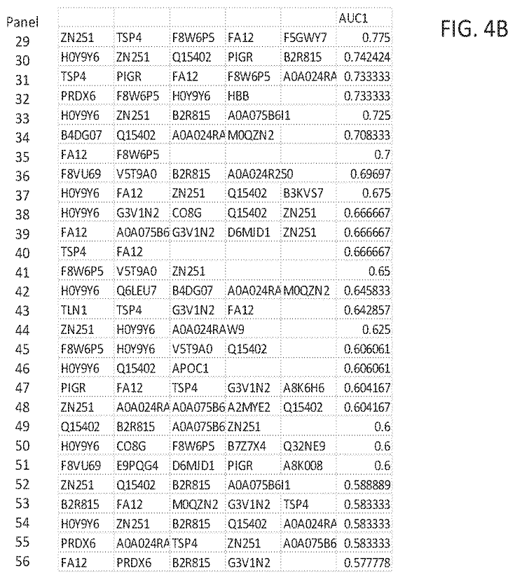

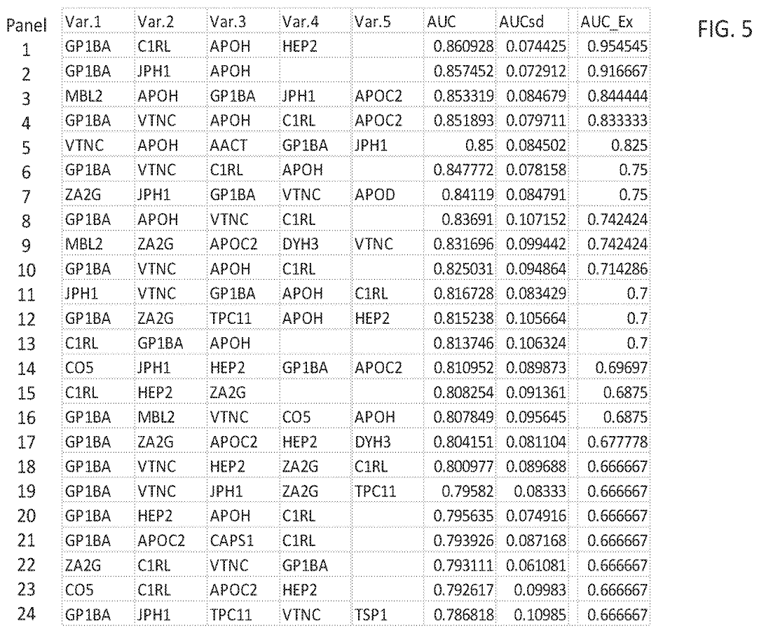

In one aspect provided herein is a method for assessing risk of preeclampsia in a pregnant subject, the method comprising: (a) preparing a microparticle-enriched fraction from a blood sample from the pregnant subject; (b) determining a quantitative measure of one or more microparticle-associated protein biomarkers in the fraction, wherein the one or more protein biomarkers are selected from: (i) a protein biomarker of Table 1; (ii) a protein biomarker of the set: A2N0U6, A0A024R8D8, B2R6L0, GP1BA, Q96TB4, A0A075B6I4, Q5NV82, E3UVQ2, E9PQG4, L0R6N9, VTNC, C1RL, MBL2, B2R815, D6MJD1, ZA2G, A0A024R9I2, TPC11, CO5, A0A024R3Z1, A8K008, B2R4C5, B4E1D8, GP112, A0A075B6H9; and (iii) a protein biomarker of the set: GP1BA, VTNC, C1RL, ZA2G, APOC2, APOH, JPH1, CO5, HEP2, TPC11, MBL2, AACT, DYH3, TSP1, CAPS1, APOD, LCAT; and (c) assessing the risk of preeclampsia based on the measure. In one embodiment, an increased amount of an up-regulated biomarker or a decreased amount of a down-regulated biomarker indicates increased risk of preeclampsia. In another embodiment, the method comprises determining a quantitative measure of a plurality of protein biomarkers selected from the protein biomarkers of Table 1. In another embodiment, the one or more protein biomarkers are selected from Table 1: Group 1, Group 2 or Group 3. In another embodiment, the one or more protein biomarkers are selected from each of a plurality of biological functions selected from immune function, cell signaling, angiogenesis, apoptosis, matrix attachment, cell function, protein metabolism, ion transport and unknown function. In another embodiment, the method comprises determining risk of severe preeclampsia wherein the biomarker or biomarkers are selected from: 0A075B6I5_HUMAN, A2MYD2_HUMAN, AL2SA_HUMAN, AR13B_HUMAN, B3AT_HUMAN, BAI1_HUMAN, BRWD3_HUMAN, C6K6H8_HUMAN, CI040_HUMAN, CPLX1_HUMAN, CPLX2_HUMAN, E5RG74_HUMAN, E9PNW5_HUMAN, HV301_HUMAN, I6Y0B1_HUMAN, J3KPJ3_HUMAN, LAC7_HUMAN, LIPA2_HUMAN, LV104_HUMAN, LV109_HUMAN, Q68D13_HUMAN, Q9UL88_HUMAN, SCRIB_HUMAN and TTC37_HUMAN. In another embodiment, the method comprises determining a quantitative measure of a plurality of protein biomarkers selected from A2N0U6, A0A024R8D8, B2R6L0, GP1BA, Q96TB4, A0A075B6I4, Q5NV82, E3UVQ2, E9PQG4, L0R6N9, VTNC, C1RL, MBL2, B2R815, D6MJD1, ZA2G, A0A024R9I2, TPC11, CO5, A0A024R3Z1, A8K008, B2R4C5, B4E1D8, GP112, and A0A075B6H9. In another embodiment, the method comprises determining a quantitative measure of a plurality of protein biomarkers selected from GP1BA, VTNC, C1RL, ZA2G, APOC2, APOH, JPH1, CO5, HEP2, TPC11, MBL2, AACT, DYH3, TSP1, CAPS1, APOD, and LCAT. In another embodiment the biomarkers comprise a panel of biomarkers selected from panels 1-29 (FIG. 3), panels 1-56 (FIGS. 4A-4B) and panels 1-24 (FIG. 5). In another embodiment wherein, the panel comprises no more than any of 10, 9, 8, 7, 6, 5, 4 or 3 protein biomarkers. In another embodiment the biomarkers consist of a panel of biomarkers selected from panels 1-29 (FIG. 3), panels 1-56 (FIGS. 4A-4B) and panels 1-24 (FIG. 5). In another embodiment the biomarkers comprise a panel of biomarkers including 5, 4, 3 or 2 biomarkers selected from A2N0U6, A0A024R8D8, B2R6L0, GP1BA and Q96TB4. In another embodiment the biomarkers comprise a panel of biomarkers including A2N0U6 and at least 1, 2, 3, or 4 of A0A024R8D8, B2R6L0, GP1BA and Q96TB4. In another embodiment the biomarkers comprise a panel of biomarkers including 6, 5, 4, 3 or 2 biomarkers selected from GP1BA, VTNC, C1RL, ZA2G, APOC2 and APOH. In another embodiment the biomarkers comprise a panel of biomarkers including GP1BA and at least 1, 2, 3, 4 or 5 of VTNC, C1RL, ZA2G, APOC2 and APOH. In another embodiment the sample is taken from the pregnant subject during the first trimester or second trimester of pregnancy. In another embodiment the sample is taken from the pregnant subject during weeks 10-12 of gestation. In another embodiment the pregnant subject is primigravida, multigravida, primiparous or multiparous. In another embodiment the pregnant subject has a singleton pregnancy or multiple pregnancy. In another embodiment the pregnant subject is asymptomatic for preeclampsia, e.g., is not hypertensive or does not have proteinuria. In another embodiment the pregnant subject has no history of preeclampsia. In another embodiment the pregnant subject has no risk factors for preeclampsia. In another embodiment the pregnant subject has chronic hypertension. In another embodiment the blood sample is plasma or serum. In another embodiment the microparticle-enriched fraction is prepared using size-exclusion chromatography. In another embodiment the size-exclusion chromatography comprises elution with water. In another embodiment the size-exclusion chromatography is performed with an agarose solid phase and an aqueous liquid phase. In another embodiment the preparing step further comprises using ultrafiltration or reverse-phase chromatography. In another embodiment the preparing step further comprises denaturation using urea, reduction using dithiothreitol, alkylation using iodoacetamine, and digestion using trypsin after the size exclusion chromatography. In another embodiment the microparticles are further purified to enrich for placental-derived exosomes or vascular endothelial-derived exosomes. In another embodiment determining a quantitative measure comprises mass spectrometry. In another embodiment determining a quantitative measure comprises liquid chromatography/mass spectrometry (LC/MS). In another embodiment mass spectrometry comprises liquid chromatography/triple quadrupole mass spectrometry. In another embodiment the mass spectrometry comprises multiple reaction monitoring. In another embodiment the mass spectrometry comprises multiple reaction monitoring, and the liquid chromatography is done using a solvent comprising acetonitrile, and/or determining comprises assigning an indexed retention time to the protein biomarkers. In another embodiment the mass spectrometry comprises multiple reaction monitoring, and the method comprises adding one or more stable isotope standard peptides to the sample before introduction into the mass spectrometer and detection comprises detecting one or a plurality of daughter ions of the stable isotope peptide standards produced by a collision cell of the mass spectrometer. In another embodiment determining the quantitative measure comprises determining a quantitative measure of a surrogate peptide of the protein biomarker. In another embodiment mass spectrometry comprises quantifying one or more stable isotope labeled standard peptides. In another embodiment MRM comprises adding one or more stable heavy isotope substituted standards corresponding to said protein biomarkers to the microparticle enriched fraction. In another embodiment wherein determining a quantitative measure comprises contacting the sample with one or more capture reagents, each capture reagent specifically binding one of the protein biomarkers, and detecting binding between the capture reagent in the protein biomarker. In another embodiment quantifying comprises performing an immunoassay. In another embodiment the immunoassay is selected from the group consisting of enzyme immunoassay (EIA), enzyme-linked immunosorbent assay (ELISA), and radioimmunoassay (RIA). In another embodiment the assessing comprises executing a classification rule, which rule classifies the subject at being at risk of preeclampsia, and wherein execution of the classification rule produces a correlation between preeclampsia or term birth with a p value of less than at least 0.05. In another embodiment the assessing comprises executing a classification rule, which rule classifies the subject at being at risk of preeclampsia, and wherein execution of the classification rule produces a receiver operating characteristic (ROC) curve, wherein the ROC curve has an area under the curve (AUC) of at least 0.6, at least 0.7, at least 0.8 or at least 0.9. In another embodiment values on which the classification rule classifies a subject further include at least one of: maternal age, maternal body mass index, primiparous, and smoking during pregnancy. In another embodiment the classification rule employs cut-off, linear regression (e.g., multiple linear regression (MLR), partial least squares (PLS) regression, principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (e.g., support vector machines). In another embodiment wherein, the classification rule is configured to have a sensitivity, specificity, positive predictive value or negative predictive value of at least 70%, least 80%, at least 90% or at least 95%. In another embodiment assessing an increased risk of preeclampsia comprises determining that the protein biomarker (if upregulated) is above or (if down regulated) is below a threshold level. In another embodiment the threshold level represents a level at least one, at least two or at least three z scores from a measure of central tendency (e.g., mean, median or mode) for the protein determined from at least 50, at least 100 or at least 200 control subjects. In another embodiment the assessing comprises comparing the measure of each protein in the panel to a reference standard. In another embodiment, the method further comprises communicating the risk of preeclampsia for a pregnant subject to a health care provider. In another embodiment, the method further comprises: (d) determining, a quantitative measure of one or more microparticle-associated protein biomarkers for preterm birth in the fraction; and (e) assessing the risk of preterm birth based on the measure.

In another aspect provided herein is a method of decreasing risk of preeclampsia for a pregnant subject and/or reducing neonatal complications of preeclampsia, the method comprising: (a) assessing risk of preeclampsia for a pregnant subject according to a method as described herein; and (b) administering a therapeutic intervention to the subject effective to decrease the risk of preeclampsia and/or reduce neonatal complications of preeclampsia. In another embodiment the therapeutic intervention is selected from the group consisting of aspirin (e.g., low dose aspirin), a corticosteroid or a medication to reduce hypertension. In another embodiment the preeclampsia treated is a later or milder form, hypertensive form or earlier or severe form.

In another aspect provided herein is a method comprising administering to a pregnant subject determined to have an increased risk of preeclampsia by a method as described herein, a therapeutic intervention effective to reduce the risk of preeclampsia or to reduce neonatal complications of preeclampsia.

In another aspect provided herein is a method of administering to a pregnant subject having an altered quantitative measure as compared to a reference standard of any one of the panels of protein biomarkers selected from panels 1-29 (FIG. 3), panels 1-56 (FIGS. 4A-4B) and panels 1-24 (FIG. 5), an effective amount of a treatment designed to reduce the risk of preeclampsia.

In another aspect provided herein is a panel comprising a plurality of substantially pure protein biomarkers or surrogate biomarkers selected from the protein biomarkers of Table 1, Table 3 or Table 4. In one embodiment, the panel further comprises a stable isotope standard peptide paired with each of the surrogate biomarkers.

In another aspect provided herein is a kit comprising one or a plurality of containers, wherein each container comprises one or more of each of a plurality of Stable Isotopic Standards, each stable isotopic standard corresponding to a surrogate peptide for a biomarker from a panel of biomarkers selected from panels 1-29 (FIG. 3), panels 1-56 (FIGS. 4A-4B) and panels 1-24 (FIG. 5).

In another aspect provided herein is a computer readable medium in tangible, non-transitory form comprising code to implement a classification rule generated by a method as described herein.

In another aspect provided herein is a system comprising: (a) a computer comprising: (i) a processor; and (II) a memory, coupled to the processor, the memory storing a module comprising: (1) test data for a sample from a subject including values indicating a quantitative measure of one or more protein biomarkers in the fraction, wherein the protein biomarkers are selected from the protein biomarkers of Table 1, Table 3 and Table 4; (2) a classification rule which, based on values including the measurements, classifies the subject as being at risk of pre-term birth, wherein the classification rule is configured to have a sensitivity of at least 75%, at least 85% or at least 95%; and (3) computer executable instructions for implementing the classification rule on the test data.

### DETAILED DESCRIPTION

### I. Introduction

Disclosed herein are methods, systems and articles useful in determining risk of developing, and for treating, preeclampsia. This includes early detection of preeclampsia (determination while the condition is sub-clinical and/or below normal threshold for detection) and determination of risk of developing preeclampsia. Certain of these relate to the detection of preeclampsia biomarkers found in microparticle-enriched fractions from the blood of pregnant women. Such biomarkers are presented in Table 1, Table 4 and Table 5.

### II. Subjects

Subjects for prediction and treatment of preeclampsia are pregnant human females. In some embodiments, the pregnant woman is in the first trimester (e.g., weeks 1-12 of gestation), second trimester (e.g., weeks 13-28 of gestation) or third trimester (e.g., weeks 29-37 of gestation) of pregnancy. In some embodiments, the pregnant woman is in early pregnancy (e.g., from 8, 9, 10, 11, 12, 13, 14, 15, 16, 17, 18, 19 or 20, but earlier than 21 weeks of gestation; from 20, 19, 18, 17, 16, 15, 14, 13, 12, 11, 10 or 9, but later than 8 weeks of gestation). In some embodiments, the pregnant woman is between 8-15 weeks of pregnancy, for example, 10-12 weeks, 8-12 weeks or 10-15 weeks. In some embodiments, the pregnant woman is in mid-pregnancy (e.g., from 21, 22, 23, 24, 25, 26, 27, 28, 29 or 30, but earlier than 31 weeks of gestation; from 30, 29, 28, 27, 26, 25, 24, 23, 22 or 21, but later than 20 weeks of gestation). In some embodiments, the pregnant woman is in late pregnancy (e.g., from 31, 32, 33, 34, 35, 36 or 37, but earlier than 38 weeks of gestation; from 37, 36, 35, 34, 33, 32 or 31, but later than 30 weeks of gestation). In some embodiments, the pregnant woman is in less than 17 weeks, less than 16 weeks, less than 15 weeks, less than 14 weeks or less than 13 weeks of gestation; from 20, 19, 18, 17, 16, 15, 14, 13, 12, 11, 10 or 9, but later than 8 weeks of gestation). The stage of pregnancy can be calculated from the first day of the last normal menstrual period of the pregnant subject.

Pregnant subjects of the methods described herein can belong to one or more classes including primiparous (no previous child brought to delivery, interchangeably referred to herein as nulliparous or parity=0) or multiparous (at least one previous child brought to at least 20 weeks of gestation, referred to interchangeably herein as parity>0, parity≥1), primigravida (first pregnancy) or multigravida (more than one pregnancy).

In some embodiments, the pregnant human subject is asymptomatic. In some embodiments, the subject may have a risk factor of preeclampsia such as high blood pressure, protein in the urine, a family history of preeclampsia, renal or connective tissue disease, obesity, advanced maternal age, or a conception with medical assistance.

### III. Sample Preparation

A sample for use in the methods of the present disclosure is a biological sample obtained from a pregnant subject. In certain embodiments, the sample is collected during a stage of pregnancy described in the preceding section. In some embodiments, the sample is a blood, saliva, tears, sweat, nasal secretions, urine, amniotic fluid or cervicovaginal fluid sample. In some embodiments, the sample is a blood sample, which in certain embodiments are serum or plasma. In some embodiments, the sample has been stored frozen (e.g., −20° C. or −80° C.).

The term “microparticle” refers to an extracellular microvesicle or lipid raft protein aggregate having a hydrodynamic diameter of about 50 to about 5000 nm. As such, the term microparticle encompasses exosomes (about 50 to about 100 nm), microvesicles (about 100 to about 300 nm), ectosomes (about 50 to about 1000 nm), apoptotic bodies (about 50 to about 5000 nm) and lipid-protein aggregates of the same dimensions.

The term “microparticle-associated protein” refers to a protein or fragment thereof that is detectable in a microparticle-enriched sample from a mammalian (e.g., human) subject. As such the term “microparticle-associated protein” is not restricted to proteins or fragments thereof that are physically associated with microparticles at the time of detection.

The term “polypeptide” as used herein refers to an amino acid polymer including peptides, polypeptides and proteins, unless otherwise specified.

The term “about” as used herein in reference to a value refers to 90% to 110% of that value. For instance, a diameter of about 1000 nm is a diameter within the range of 900 nm to 1100 nm.

Biomarkers for preeclampsia can be derived from microparticles. Microparticles can be isolated from blood (e.g., serum or plasma) by size exclusion chromatography. The elution buffer can be, for example, a buffered solution such as PBS, a non-buffered solution, water, or de-ionized water. The high molecular weight fraction can be collected to obtain a microparticle-enriched sample. Proteins within the microparticle-enriched sample are then extracted before digestion with a proteolytic enzyme such as trypsin to obtain a digested sample comprising a plurality of peptides. The digested sample is then subjected to a peptide purification/concentration step before analysis to obtain a proteomic profile of the sample, e.g., by liquid chromatography and mass spectrometry. In some embodiments, the purification/concentration step comprises reverse phase chromatography (e.g., ZIPTIP pipette tip with 0.2 μL C18 resin, from Millipore Corporation, Billerica, MA).

In certain embodiments, the exosomes are placental-derived exosomes or endothelial-derived exosomes. Such exosomes can be isolated using capture agents, such as antibodies, against surface markers for these cells of origin. For example, placental-derived exosomes can be isolated using antibodies directed to CD34, CD44 or leukemia inhibitory factor (LIF). Endothelial-derived exosomes can be isolated using antibodies directed to ICAM or VCAM.

Provided herein are compositions of matter comprising one or a plurality of preeclampsia biomarkers in substantially pure form. The biomarkers can be mixed in a container, or can be physically separated, for example, through attachment to solid supports at different addressable locations. As used herein, a chemical entity, such as a polynucleotide or polypeptide, is “substantially pure” if it is the predominant chemical entity of its kind in a composition. This includes the chemical entity representing more than 50%, more than 80%, more than 90% or more than 95% or of the chemical entities of its kind in the composition. A chemical entity is “essentially pure” if it represents more than 98%, more than 99%, more than 99.5%, more than 99.9%, or more than 99.99% of the chemical entities of its kind in the composition. Chemical entities which are essentially pure are also substantially pure.

### IV. Biomarker Detection

### A. Biomarkers

As used herein, the term “biomarker” refers to a biological molecule, the presence, form or amount of which exhibits a statistically significant difference between two states. Accordingly, biomarkers are useful, alone or in combination, for classifying a subject into one of a plurality of groups. Biomarkers may be naturally occurring or non-naturally occurring. For example, a biomarker may be naturally occurring protein or a non-naturally occurring fragment of a protein. Fragments of a protein can function as a proxy or surrogate peptide for the protein or as stand-alone biomarkers.

Provided herein are polypeptide biomarkers for risk of preeclampsia. Biomarkers for preeclampsia are presented in Table 1, Table 3 and Table 4. Panels of biomarkers for risk of preeclampsia are presented in FIG. 3, FIGS. 4A and 4B, and FIG. 5.

The biomarkers can be detected using de novo sequencing of proteins from microparticles isolated from a sample (e.g. blood) taken from a pregnant woman. Proteins can be sequenced by mass spectrometry, e.g., single or double (MS/MS) mass spectrometry. Both parent proteins and peptide fragments of parent proteins are useful as biomarkers of preeclampsia. Unless otherwise specified, a named protein biomarker encompasses detection by surrogate, e.g., fragments of the protein.

Proteins, e.g., peptides, detected by mass spectrometry are analyzed to identify those that are up-regulated (increased in amounts) or down-regulated (decreased in amounts) compared with controls. Proteins showing statistically significant differential expression are further analyzed to identify the parent protein. Such proteins can be identified in a protein database such as SwissProt.

In certain embodiments, biomarkers are analyzed as a panel comprising a plurality of the biomarkers. A panel can exist as a conceptual grouping, as a composition of matter (e.g., comprising purified biomarker polypeptides, or as an article, such as solid support attached to a capture reagent such as an antibody, further bound to the biomarker. The solid support can be, for example, one or more solid particles, such as beads, or a chip in which biomarkers are attached in an array format.

In certain embodiments, biomarkers can be comprised in a composition in which the peptide biomarker is paired with and a stable isotopic standard of the peptide. Such compositions are useful for detection in multiple reaction monitoring mass spectrometry.

For purposes of mass spectrometry, proteins can be detected intact, or through fragmentation, e.g., in multiple reaction monitoring (MRM). In such cases, proteins can be fragmented proteolytically before analysis. Proteolytic fragmentation includes both chemical and enzymatic fragmentation. Chemical fragmentation includes, for example, treatment with cyanogen bromide. Enzymatic fragmentation includes, for example, digestion with proteases such as trypsin, chymotrypsin, LysC, ArgC, GluC, LysN and AspN. Detection of these protein fragments, or fragmented forms of them produced in mass spectrometry, can function as surrogates for the full protein.

1. Biomarkers Identified from Initial Analysis

Initial statistical analysis of microsomal-associated proteins identified the biomarkers of Table 1. Table 1 indicates the relative rank (“Rank”) of the biomarker's discriminating power (1, 2 or 3), whether the biomarker also functions in classifying extreme cases of PE (“Also found in extreme phenotype”), the full name of the protein biomarker, the ratio of the amount of the biomarker in cases versus controls, and the differential expression p value. As regards ratio, a ratio greater than 1 indicates that the marker is up-regulated in PE, while a ratio less than 1 indicates the biomarker is down-regulated in PE. Extreme preeclampsia, also referred to as severe preeclampsia, is characterized by one or more of headaches, blurred vision, inability to tolerate bright light, fatigue, nausea/vomiting, urinating small amounts, pain in the upper right abdomen, shortness of breath, and tendency to bruise easily.

Biomarkers used for predictions of preeclampsia can be one or more than one biomarker selected from all of the biomarkers in Table 1, below, or one or more than one biomarker selected from any rank group of the biomarkers in Table 1. Biomarkers selected may all be up-regulated, all be down-regulated or a combination of both up and down regulated biomarkers.

In certain embodiments, the biomarkers are selected from: 0A075B6I5_HUMAN, A2MYD2_HUMAN, AL2SA_HUMAN, AR13B_HUMAN, B3AT_HUMAN, BAI1_HUMAN, BRWD3_HUMAN, C6K6H8_HUMAN, CI040_HUMAN, CPLX1_HUMAN, CPLX2_HUMAN, E5RG74_HUMAN, E9PNW5_HUMAN, HV301_HUMAN, I6Y0B1_HUMAN, J3KPJ3_HUMAN, LAC7_HUMAN, LIPA2_HUMAN, LV104_HUMAN, LV109_HUMAN, Q68D13_HUMAN, Q9UL88_HUMAN, SCRIB_HUMAN and TTC37_HUMAN. Such biomarkers maybe correlated with a severe form of preeclampsia.

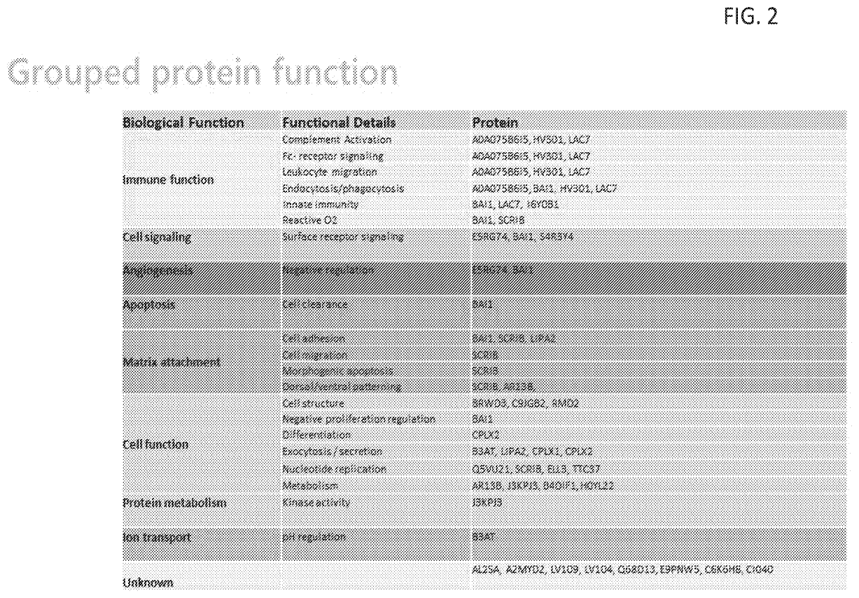

FIG. 2 shows biological functions with which biomarkers for increased risk of preeclampsia are associated. These biological functions include immune function, cell signaling, angiogenesis, apoptosis, matrix attachment, cell function, protein metabolism and ion transport. Biomarkers for proteins of unknown biological function also are shown. In certain embodiments, at least one biomarker from each of a plurality (e.g., at least two, at least three, at least 3, at least 4, at least 5, at least 6, at least 7 or at least 8) of different biological functions can be measured. This can include measuring at least biomarker for a protein of unknown biological function as well.

### 2. Biomarkers Identified With Machine Learning

Using machine learning on data produced by HRAM mass spectrometry analysis, other well-performing biomarkers were discovered, presented in Table 3 and Table 4. Panels using these biomarkers are presented in FIG. 3, and FIGS. 4A and 4B. In another embodiment the proteins biomarkers can be 1, 2, 3, 4, 5, 6 or more biomarkers selected from A2N0U6, A0A024R8D8, B2R6L0, GP1BA, Q96TB4, A0A075B6I4, Q5NV82, E3UVQ2, E9PQG4, L0R6N9, VTNC, C1RL, MBL2, B2R815, D6MJD1, ZA2G, A0A024R9I2, TPC11, CO5, A0A024R3Z1, A8K008, B2R4C5, B4E1D8, GP112, and A0A075B6H9. Alternatively, a panel can include no more than any of 6, 5, 4, 3, or 2 biomarkers selected from this group.

Protein biomarkers useful in the methods described herein include panels of biomarkers. A panel of biomarkers can comprise proteins from a panel selected from panels 1-29 of FIG. 3. That is, a panel can include biomarkers from a panel selected from panels 1-29 of FIG. 3 and other biomarkers in addition. In another embodiment, a panel of biomarkers can consist of a panel of biomarkers selected from panels 1-29 of FIG. 3. That is, the panel includes only the biomarkers identified in the panel specified.

Other panels of biomarkers include panels comprising protein biomarkers from a panel selected from panels 1-56 of FIGS. 4A-4B. In another embodiment the panel consists of protein biomarkers from a panel selected from panels 1-56 of FIGS. 4A-4B.

In other embodiments, the biomarkers comprise a panel of biomarkers including 5, 4, 3 or 2 biomarkers selected from A2N0U6, A0A024R8D8, B2R6L0, GP1BA and Q96TB4.

In other embodiments, the biomarkers comprise a panel of biomarkers including A2N0U6 and at least 1, 2, 3, or 4 of A0A024R8D8, B2R6L0, GP1BA and Q96TB4.

### 3. Biomarkers Identified After Curation

Biomarkers identified in the previous machine learning operation were curated against the STRING protein database. Proteins either not included in the STRING database or identified as having fewer than four interactions with other proteins in the database were removed. The remaining proteins had a known biological function. Data relating to the remaining proteins was for the subject to machine learning. Best performing protein biomarkers were identified and presented in Table 5 and Table 6. Best performing panels including these protein biomarkers are presented in FIG. 5.

Accordingly, in another embodiment protein biomarkers for determining risk of preeclampsia can be 1, 2, 3, 4, 5, 6 or more biomarkers selected from GP1BA, VTNC, C1RL, ZA2G, APOC2, APOH, JPH1, CO5, HEP2, TPC11, MBL2, AACT, DYH3, TSP1, CAPS1, APOD, and LCAT. Alternatively, a panel can include no more than any of 6, 5, 4, 3, or 2 biomarkers selected from this group.

A panel of biomarkers can comprise proteins from a panel selected from panels 1-24 of FIG. 5. In another embodiment the panel consists of protein biomarkers from a panel selected from panels 1-24 of FIG. 5.

In other embodiments, the biomarkers comprise a panel of biomarkers including 6, 5, 4, 3 or 2 biomarkers selected from GP1BA, VTNC, C1RL, ZA2G, APOC2 and APOH.

In other embodiments, the biomarkers comprise a panel of biomarkers including GP1BA and at least 1, 2, 3, 4 or 5 of VTNC, C1RL, ZA2G, APOC2 and APOH.

### 4. Methods of Detection

Biomarkers can be detected and quantified by any method known in the art. This includes, without limitation, immunoassay, chromatography, mass spectrometry, electrophoresis and surface plasmon resonance.

Detection of a biomarker includes detection of an intact protein, or detection of surrogate for the protein, such as a fragment.

Immunoassay methods include, for example, radioimmunoassay, enzyme-linked immunosorbent assay (ELISA), sandwich assays and Western blot, immunoprecipitation, immunohistochemistry, immunofluorescence, antibody microarray, dot blotting, and FACS.

Chromatographic methods include, for example, affinity chromatography, ion exchange chromatography, size exclusion chromatography/gel filtration chromatography, hydrophobic interaction chromatography and reverse phase chromatography, including, e.g., HPLC.

### 5. Mass Spectrometry

In some embodiments, detecting the level (e.g., including detecting the presence) of a microparticle-associated protein is accomplished using a liquid chromatography/mass spectrometry (LCMS)-based proteomic analysis. In an exemplary embodiment the method involves subjecting a sample to size exclusion chromatography and collecting the high molecular weight fraction (e.g., by size-exclusion chromatography) to obtain a microparticle-enriched sample. The microparticle-enriched sample is then disrupted (using, for example, chaotropic agents, denaturing agents, reducing agents and/or alkylating agents) and the released contents subjected to proteolysis. The disrupted exosome preparation, containing a plurality of peptides, is then processed using the tandem column system described herein prior to peptide analysis by mass spectrometry, to provide a proteomic profile of the sample. The methods disclosed herein avoid the necessity of protein concentration/purification, buffer exchange and liquid chromatography steps associated with previous methods.

Proteins in a sample can be detected by mass spectrometry. Mass spectrometers typically include an ion source to ionize analytes, and one or more mass analyzers to determine mass. Mass analyzers can be used together in tandem mass spectrometers. Ionization methods include, among others, electrospray or laser desorption methods. Mass analyzers include quadrupoles, ion traps, time-of-flight instruments and magnetic or electric sector instruments. In certain embodiments, the mass spectrometer is a tandem mass spectrometer (e.g., “MS-MS”) that uses a first mass analyzer to select ions of a certain mass and a second mass analyzer to analyze the selected ions. One example of a tandem mass spectrometer is a triple quadrupole instrument, the first and third quadrupoles act as mass filters, and an intermediate quadrupole functions as a collision cell. Mass spectrometry also can be coupled with up-stream separation techniques, such as liquid chromatography or gas chromatography. So, for example, liquid chromatography coupled with tandem mass spectrometry can be referred to as “LC-MS-MS”.

Mass spectrometers useful for the analyses described herein include, without limitation, Altis™ quadrupole, Quantis™ quadrupole, Quantiva™ or Fortis™ triple quadrupole from ThermoFisher Scientific, and the QSight™ Triple Quad LC/MS/MS from Perkin Elmer.

Generally, any mass spectrometric (MS) technique that can provide precise information on the mass of peptides, and preferably also on fragmentation and/or (partial) amino acid sequence of selected peptides (e.g., in tandem mass spectrometry, MS/MS; or in post source decay, TOF MS), can be used in the methods and compositions disclosed herein. Suitable peptide MS and MS/MS techniques and systems are known in the art (see, e.g., Methods in Molecular Biology, vol. 146: “Mass Spectrometry of Proteins and Peptides”, by Chapman, ed., Humana Press 2000; Kassel & Biemann (1990) Anal. Chem. 62:1691-1695; Methods Enzymol 193:455-79; or Methods in Enzymology, vol. 402: “Biological Mass Spectrometry”, by Burlingame, ed., Academic Press 2005) and can be used in practicing the methods disclosed herein. Accordingly, in some embodiments, the disclosed methods comprise performing quantitative MS to measure one or more peptides. Such quantitative methods can be performed in an automated (Villanueva, et al., Nature Protocols (2006) 1(2):880-891) or semi-automated format. In particular embodiments, MS can be operably linked to a liquid chromatography device (LC-MS/MS or LC-MS) or gas chromatography device (GC-MS or GC-MS/MS).

Selected reaction monitoring is a mass spectrometry method in which a first mass analyzer selects a polypeptide of interest (precursor), a collision cell fragments the polypeptide into product fragments and one or more of the fragments is detected in a second mass analyzer. The precursor and product ion pair is called an SRM “transition”. The method is typically performed in a triple quadrupole instrument. When multiple fragments of a polypeptide are analyzed, the method is referred to as Multiple Reaction Monitoring Mass Spectrometry (“MRM-MS”).

Typically, protein samples are digested with a proteolytic enzyme, such as trypsin, to produce peptide fragments. Heavy isotope labeled analogues of certain of these peptides are synthesized as standards. These standards are referred to as Stable Isotopic Standards or “SIS”. SIS peptides are mixed with a protease-treated sample. The mixture is subjected to triple quadrupole mass spectrometry. Peptides corresponding to the daughter ions of the SIS standards and the target peptides are detected with high accuracy, in either the time domain or the mass domain. Usually, a plurality of the daughter ions is used to unambiguously identify the presence of a parent ion, and one of the daughter ions, usually the most abundant, is used for quantification. SIS peptides can be synthesized to order, or can be available as commercial kits from vendors such as, for example, e.g., ThermoFisher (Waltham, MA) or Biognosys (Zurich, Switzerland).

As used herein, the terms “multiple reaction monitoring (MRM)” or “selected reaction monitoring (SRM)” refer to a MS-based quantification method that is particularly useful for quantifying analytes that are in low abundance. In an SRM experiment, a predefined precursor ion and one or more of its fragments are selected by the two mass filters of a triple quadrupole instrument and monitored over time for precise quantification. Multiple SRM precursor and fragment ion pairs can be measured within the same experiment on the chromatographic time scale by rapidly toggling between the different precursor/fragment pairs to perform an MRM experiment. A series of transitions (precursor/fragment ion pairs) in combination with the retention time of the targeted analyte (e.g., peptide or small molecule such as chemical entity, steroid, hormone) can constitute a definitive assay. A large number of analytes can be quantified during a single LC-MS experiment. The term “scheduled,” or “dynamic” in reference to MRM or SRM, refers to a variation of the assay wherein the transitions for a particular analyte are only acquired in a time window around the expected retention time, significantly increasing the number of analytes that can be detected and quantified in a single LC-MS experiment and contributing to the selectivity of the test, as retention time is a property dependent on the physical nature of the analyte. A single analyte can also be monitored with more than one transition. Finally, the assay can include standards that correspond to the analytes of interest (e.g., peptides having the same amino acid sequence as that of analyte peptides), but differ by the inclusion of stable isotopes. Stable isotopic standards (SIS) can be incorporated into the assay at precise levels and used to quantify the corresponding unknown analyte. Additional levels of specificity are contributed by the co-elution of the unknown analyte and its corresponding SIS, and by the properties of their transitions (e.g., the similarity in the ratio of the level of two transitions of the analyte and the ratio of the two transitions of its corresponding SIS).

Accordingly, detection of a protein target by MRM-MS involves detection of one or more peptide fragments of the protein, typically through detection of a stable isotope standard peptide against which the peptide fragment is compared. Typically, an SIS will, itself, be fragmented in a collision cell as the original digested fragment, and one or more of these fragments is detected by the mass spectrometer.

Mass spectrometry assays, instruments and systems suitable for biomarker peptide analysis can include, without limitation, matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; electrospray ionization mass spectrometry (ESI-MS); ESI-MS/MS; ESI-MS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS) n; ion mobility spectrometry (IMS); inductively coupled plasma mass spectrometry (ICP-MS) atmospheric pressure photoionization mass spectrometry (APPI-MS); APPI-MS/MS; and APPI-(MS)n. Peptide ion fragmentation in tandem MS (MS/MS) arrangements can be achieved using techniques known in the art, such as, e.g., collision induced dissociation (CID). As described herein, detection and quantification of biomarkers by mass spectrometry can involve multiple reaction monitoring (MRM), such as described, inter alia, by Kuhn et al. (2004) Proteomics 4:1175-1186. Scheduled multiple-reaction-monitoring (Scheduled MRM) mode acquisition during LC-MS/MS analysis enhances the sensitivity and accuracy of peptide quantitation. Anderson and Hunter (2006) Mol. Cell. Proteomics 5(4):573-588. Mass spectrometry-based assays can be advantageously combined with upstream peptide or protein separation or fractionation methods, such as, for example, with the tandem column system described herein.

### V. Methods of Assessing Risk of Preeclampsia

The phrase “increased risk of preeclampsia” as used herein indicates that a pregnant subject has a greater likelihood of developing preeclampsia than a general population of subjects at the same stage of pregnancy, optionally compared with a population sharing one or more demographic or risk factors. These may include, for example, age, status/result of prior pregnancy, hypertension, protein in urine, race/ethnicity, medical history, prior pregnancy history, smoking/drug history, and the like. For example, a test may indicate that a woman at 10-12 weeks of pregnancy has a higher risk of developing preeclampsia than a general or control population of woman at 10-12 weeks or pregnancy.

Provided herein are methods of assessing risk for preeclampsia, for example, classifying a pregnant human female as at increased risk of preeclampsia. The methods can involve determining a quantitative measure of one or a plurality of the biomarkers in Table 1, and correlating the measure to risk of preeclampsia. For example, one can use 2, 3, 4, 5, 6 or more, or, no more than 2, 3, 4, 5, 6, biomarkers in the determination. In general, measurement of a relatively increased amount of an up-regulated biomarker or a relatively decreased amount of a down-regulated biomarker correlated with increased risk of preeclampsia. Alternatively, determination is based on a classification algorithm that may employ non-linear and/or hyperdimensional methods.

In certain embodiments, biomarkers are used to differentiate between PE subgroups such as (i) PE, later/milder form vs, (ii) PE/hypertension, earlier/severe form.

In certain embodiments, the methods further comprise performing uterine artery Doppler ultrasound or measuring maternal blood pressure.

Methods of assessing risk of preeclampsia can involve classifying a subject as at increased risk of preeclampsia based on information including at least a quantitative measure of at least one biomarker of this disclosure.

Classifying can employ a classification algorithm or model determined by statistical analysis and/or machine learning.

### B. Statistical Analysis

Typically, analysis involves statistical analysis of a sufficiently large number of samples to provide statistically meaningful results. Any statistical method known in the art can be used for this purpose. Such methods, or tools, include, without limitation, correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression, stepwise regression, ridge regression, lasso regression, elasticnet regression) or non-parametric analysis (e.g., Wilcoxon rank-sum test, Wilcoxon sign-rank test, sign test). Such tools are included in commercially available statistical packages such as MATLAB, JMP Statistical Software and SAS. Such methods produce models or classifiers which one can use to classify a particular biomarker profile into a particular state.

Statistical analysis can be operator implemented or implemented by machine learning.

### C. Machine Learning

Many types of classification algorithms are suitable for this purpose, including linear and non-linear models, e.g., processes such as CART-classification and regression trees), artificial neural networks such as back propagation networks, discriminant analyses (e.g., Bayesian classifier or Fischer analysis), logistic classifiers, and support vector classifiers (e.g., support vector machines). Certain classifiers, such as cut-offs, can be executed by human inspection. Other classifiers, such as multivariate classifiers, can require a computer to execute the classification algorithm.

Classification algorithms, also referred to as models, can be generated by mathematical analysis, including by machine learning algorithms that perform analysis of datasets of biomarker measurements derived from subjects classed into one or another group. Many machine learning algorithms are known in the art, including those that generate the types of classification algorithms above.

Diagnostic tests are characterized by sensitivity (percentage classified as positive that are true positives) and specificity (percentage classified as negative that are true negatives). The relative sensitivity and specificity of a diagnostic test can involve a trade-off-higher sensitivity can mean lower specificity, while higher specificity can mean lower sensitivity. These relative values can be displayed on a receiver operating characteristic (ROC) curve. The diagnostic power of a set of variables, such as biomarkers, is reflected by the area under the curve (AUC) of an ROC curve.

In some embodiments, the classifiers of this disclosure have a sensitivity of at least 85%, at least 90%, at least 95%, at least 98%, or at least 99%. Classifiers of this disclosure have an AUC of at least 0.6, at least 0.7, at least 0.8, at least 0.9 or at least 0.95.

Classification can be based on a measurement of a biomarker being above or below a selected cutoff level. In certain embodiments, a cutoff value is obtained by measuring biomarker levels in a plurality of positive and negative reference samples, e.g., at least 10, 20, 50, 100 or 200 samples of each type. A cutoff can be established with respect to a measure of central tendency, such as mean, median or mode in the negative samples. A measure of deviation from this measure of central tendency can be used to set the cutoff. For example, the cutoff can be set based on variance or standard deviation. For example, the cutoff can be based on Z score, that is, a number of standard deviations above a mean of normal samples, for example one standard deviation, two standard deviations, three standard deviations or four standard deviations. For example, cutoff values can be selected so that the diagnostic test has at least 80%, 90%, 95%, 98%, 99%, 99.5%, or 99.9% sensitivity, specificity and/or positive predictive value.

Numerically, an increased risk is associated with an odds ratio of over 1.0, preferably over 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 for preeclampsia.

In other embodiments, further provided herein is the measurement of biomarkers for pre-term birth from the same microparticle-enriched fraction used for measurement of preeclampsia biomarkers, and their use for predicting risk of preterm birth. Biomarkers for preterm birth are described, for example, in US publication 2015-0355188 (“Biomarkers for preterm birth”) and in International Application WO 2017/096405 (“Use of circulating microparticles to stratify risk of preterm birth”).

### VI. Methods of Treating Subjects at Risk for Preeclampsia

Methods of treating pregnant subjects suffering from or at increased risk of preeclampsia include administration of therapeutic interventions useful in treating preeclampsia. This includes, for example, administration of pharmaceutical drugs to treat elevated blood pressure, administration of drugs such as aspirin (e.g., low dose aspirin, e.g., 80 mg.), administration of statins and intensified monitoring for symptoms of preeclampsia. It also includes administration of targeted inhibitors of complement activation.

### VII. Kits

In another embodiment, provided herein are kits of reagents useful in detecting biomarkers for increased risk of preeclampsia in a sample. Reagents capable of detecting protein biomarkers include but are not limited to antibodies. Antibodies capable of detecting protein biomarkers are also typically directly or indirectly linked to a molecule such as a fluorophore or an enzyme, which can catalyze a detectable reaction to indicate the binding of the reagents to their respective targets.

In some embodiments, the kits further comprise sample processing materials comprising a high molecular weight gel filtration composition (e.g., agarose such as SEPHAROSE) in a low volume (e.g., 1 ml, 3 ml, 5 ml, 10 ml) vertical column for rapid preparation of a microparticle-enriched sample from plasma. For instance, the microparticle-enriched sample can be prepared at the point of care before freezing and shipping to an analytical laboratory for further processing.

In some embodiments, the kits further comprise instructions for assessing risk of preeclampsia. As used herein, the term “instructions” refers to directions for using the reagents contained in the kit for detecting the presence (including determining the expression level) of a protein(s) of interest in a sample from a subject. The proteins of interest may comprise one or more biomarkers of preeclampsia. In some embodiments, the instructions further comprise the statement of intended use required by the U.S. Food and Drug Administration (FDA) in labeling in vitro diagnostic products. The FDA classifies in vitro diagnostics as medical devices and required that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; and 8) Any additional information regarding the in vitro diagnostic product requested that is necessary for the FDA to make a substantial equivalency determination.

In another embodiment, a kit comprises a container containing one or a plurality of stable isotope standard (SIS) peptides corresponding to peptide biomarkers, e.g., peptides produced from protease (e.g., trypsin) digestion of biomarker proteins. In another embodiment, a majority or all of the SIS peptides correspond to the biomarker peptides. In another embodiment, the kit further comprises the biomarker peptides which the SIS peptides correspond.

### VIII. Systems

Provided herein also is a system comprising a computer comprising a processor and memory. The computer can be configured to receive into memory quantitative measures of one or more biomarkers has provided herein measured from a sample. The memory can include computer readable instructions which, when executed, classify the sample as at risk of preeclampsia or not at risk of preeclampsia. The computer system can be operatively coupled to a computer network with the aid of a communications interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The system can include a first computer connected with a second computer through a communications network, such as, a high-speed transmission network including, without limitation, Digital Subscriber Line (DSL), Cable Modem, Fiber, Wireless, Satellite and, Broadband over Powerlines (BPL). Accordingly, results providing classification of a sample as at increased risk or as not at increased risk of preeclampsia can be transmitted from a transmitting computer to a remote receiving computer, such as located at the office of a healthcare provider or to a mobile device, such as a smart phone.

### EXAMPLES

Abbreviations: AUC (area under curve); CI (confidence interval); CMP (circulating microparticles); FDR (false discovery rate); LC (liquid chromatography); LMP (last menstrual period); MRM (multiple reaction monitoring); MS (mass spectrometry); ROC (receiver operating characteristic); SEC (size exclusion chromatography).

Introduction: The canonical view of preeclampsia (PE) pathophysiology has been as an aberration of trophoblastic invasion/function at the end of the first trimester. This study shows that a unique pattern of circulating microparticle (CMP) proteins can, at this gestational age, distinguish women who develop PE; these patterns will associate with unique and early dysfunction at the maternal systemic and uteroplacental levels.

Objective: Circulating microparticles (CMPs) are nanosized lipid bilayer particles secreted by most types of cells and are increasingly appreciated as powerful mediators of both cellular communication and behavior. Prior work has associated increases in the concentrations of circulating CMP among women diagnosed with preeclampsia. Because preeclampsia is characterized by aberrant trophoblastic interactions with maternal uterine and systemic physiology at the end of the first trimester, analysis of CMP-associated proteins is expected to engender more information than circulating proteins in the blood; thus, CMPs are amenable to analysis long before the clinical presentation of preeclampsia. Patterns of CMP associated proteins sampled at a median of 12 weeks gestation are expected to differ in women who go on to develop preeclampsia versus those who have uncomplicated pregnancies.

Design: A matched case-control study of singleton pregnancies was performed. To minimize ascertainment bias and potential batch processing effects, samples were randomly selected from the prospectively collected and stored (−80° C.) EDTA plasma samples in the ongoing birth cohort that was run.

### Example 1: Isolation of Circulating Exosomes/Microparticles Biomarkers in Samples Obtained Between 10-12 Weeks Gestation.

This example describes a retrospective study on PE patients that use blood (e.g., plasma and/or serum) samples. This study is a nested, case-controlled, retrospective analysis of proteomic biomarkers detected from frozen maternal plasma samples. All samples are collected under IRB-approved protocols and all patients have been consented for research purposes. Inclusion criteria for sample collection include donations from normal, healthy, asymptomatic women with singleton gestations at two time points: 10 weeks gestation (±2 wks) and 24 weeks gestation (±2 wks). A total of 150 de-identified and blinded plasma samples (75 subjects at two time points, with 25 subjects experiencing PE in this pregnancy and 50 normal, healthy, pregnancy subjects as controls) stored in a repository are transported overnight on dry ice to an analytical laboratory and stored at −80° C.

Methods: Obstetrical outcomes in 25 singleton pregnancies with prospectively collected plasma samples obtained between 10-12 weeks were validated by physician reviewers for PE<35 weeks. These were matched to 50 uncomplicated singleton term deliveries. Controls were matched on gestational age at sampling (+/−2 weeks). CMPs from these specimens were isolated via size exclusion chromatography and analyzed using global proteome profiling based on HRAM mass spectrometry. After peptides and proteins were identified and quantified and resulting AUC ratios were used to determine differential expression between cases and controls. The identified proteins were subjected to protein complex expansion to identify meaningful pathways/interactions. Biological relevance was examined using gene ontogeny (GO) terms.

Sample Preparation. Size exclusion chromatography with buffers and workflows are used for optimal sample preparation and compatibility with mass spectrometer analysis. Alternative sample preparation methods may be coupled with buffer/workflow modifications that are optimized for other analytic approaches; or with new enrichment measures designed to sub-select exosomes originating from different tissues and organs (i.e. placental derived exosomes, or vascular endothelial derived exosomes).

Microparticles are enriched by Size Exclusion Chromatography (SEC) and isocratically eluted using water (RNAse free, DNAse free, distilled water). Briefly, PD-10 columns (GE Healthcare Life Sciences) are packed with 10 mL of 2% Agarose Bead Standard (pore size 50-150 μm) from ABT (Miami, FL), washed and stored at 4° C. for a minimum of 24 hrs and no longer than 3 days prior to use. On the day of use columns are again washed and 1 mL of thawed neat plasma sample is applied to the column. That is, the plasma samples are not filtered, diluted or treated prior to SEC.

The circulating microparticles are captured in the column void volume, partially resolved from the high abundant protein peak. One aliquot of the pooled CMP column fraction from each clinical specimen, containing 200 μg of total protein (determined by BCA) is used for further analysis.

More specifically, CMP's were isolated via size exclusion chromatography. Data were analyzed using global proteome profiling based on HRAM mass spectrometry (“high-resolution, accurate-mass mass spectrometry”). Exosomal protein was digested with trypsin and then analyzed using a Orbitrap Fusion™ Lumos™ Tribrid™ Mass Spectrometer, made by ThermoFisher Scientific. This high mass resolution system is particularly useful for analyzing complex mixtures, such as from exosomes. This methodology is useful when trying to detect peptides at low concentration in a highly complex background of peptides and other molecules.

### Example 2: Differential Expression of Proteins in Circulating Exosomes/Microparticles Between 10-12 Weeks Gestation in Pregnancies that Develop Preeclampsia.

This example shows that a unique pattern of circulating microparticle (CMP) proteins, at 10-12 weeks gestational age, distinguishes women who develop PE; these patterns associate with unique and early dysfunction at the maternal systemic and uteroplacental levels.

Results: Cases and controls did not differ by mean age (32 vs. 31; p=0.50), percent non-white (44 vs 54; p=0.38), percent nulliparous (24 vs. 28; p=0.79) but did differ on percent chronic hypertension (12 vs. 0; p=0.01) and percent prior PE (28 vs. 6; p=0.01). Untargeted analysis identified >600 unique proteins present in both sample sets at 10-12 weeks. With a FDR of 0.1, 51 proteins exhibited differential expression in cases vs. controls.

Biomarkers for preeclampsia are presented in Table 1.

TABLE 1

| | Also Found | | Case to | Differential |
|---|---|---|---|---|
| | In Extreme | | Control | Expression |
| Group | Phenotype | Full Name | Ratio | pValue |
| 1 | 1 | tr\|A0A075B6I5\|A0A075B6I5_HUMAN | 1.22 | 0.041999999 |
| | | Protein IGLV1-51 (Fragment) | | |
| 1 | 1 | tr\|A2MYD2\|A2MYD2_HUMAN V1-19 | 1.22 | 0.041999999 |
| | | protein (Fragment) | | |
| 1 | 1 | tr\|J3KPJ3\|J3KPJ3_HUMAN | 0.778 | 0.019400001 |
| | | Calcium/calmodulin-dependent protein | | |
| | | kinase kinase 1 | | |
| 1 | 1 | sp\|O75334\|LIPA2_HUMAN Liprin- | 0.778 | 0.039099999 |
| | | alpha-2 | | |
| 1 | 1 | sp\|P01702\|LV104_HUMAN Ig lambda | 1.22 | 0.041999999 |
| | | chain V-I region NIG-64 | | |
| 1 | 1 | sp\|P06888\|LV109_HUMAN Ig lambda | 1.22 | 0.041999999 |
| | | chain V-I region EPS | | |
| 1 | 0 | sp\|P01023\|A2MG_HUMAN Alpha-2- | 0.811 | 0.0306 |
| | | macroglobulin | | |
| 1 | 0 | tr\|B3KXX0\|B3KXX0_HUMAN cDNA | 0.796 | 0.040899999 |
| | | FLJ46242 fis, clone TESTI4018506, highly | | |
| | | similar to Syntaxin-binding protein 5 | | |
| 1 | 0 | sp\|P05156\|CFAI_HUMAN Complement | 1.26 | 0.018300001 |
| | | factor I | | |
| 1 | 0 | sp\|P01031\|CO5_HUMAN Complement C5 | 0.894 | 0.043900002 |
| 1 | 0 | tr\|Q14DD4\|Q14DD4_HUMAN Syntaxin | 0.796 | 0.040899999 |
| | | binding protein 5 (Tomosyn) | | |
| 1 | 0 | tr\|Q3LIE1\|Q3LIE1_HUMAN Putative | 0.796 | 0.040899999 |
| | | uncharacterized protein Nbla04300 | | |
| | | (Fragment) | | |
| 1 | 0 | tr\|Q59GS8\|Q59GS8_HUMAN | 0.894 | 0.043900002 |
| | | Complement component 5 variant | | |
| | | (Fragment) | | |
| 1 | 0 | tr\|Q6LAM1\|Q6LAM1_HUMAN Heavy | 1.26 | 0.018300001 |
| | | chain of factor I (Fragment) | | |
| 1 | 0 | tr\|Q8WW88\|Q8WW88_HUMAN CFI | 1.26 | 0.018300001 |
| | | protein | | |
| 1 | 0 | sp\|Q5T5C0\|STXB5_HUMAN Syntaxin- | 0.796 | 0.040899999 |
| | | binding protein 5 | | |
| 2 | 1 | sp\|Q3SXY8\|AR13B_HUMAN ADP- | 0.764 | 0.00512 |
| | | ribosylation factor-like protein 13B | | |
| 2 | 1 | sp\|P02730\|B3AT_HUMAN Band 3 anion | 1.22 | 0.047800001 |
| | | transport protein | | |
| 2 | 1 | sp\|O14514\|BAI1_HUMAN Brain-specific | 0.717 | 0.00332 |
| | | angiogenesis inhibitor 1 | | |
| 2 | 1 | sp\|Q6RI45\|BRWD3_HUMAN | 0.728 | 0.0124 |
| | | Bromodomain and WD repeat-containing | | |
| | | protein 3 | | |
| 2 | 1 | tr\|C6K6H8\|C6K6H8_HUMAN MHC | 0.782 | 0.0107 |
| | | class I antigen | | |
| 2 | 1 | sp\|Q8IXQ3\|CI040_HUMAN | 1.21 | 0.032299999 |
| | | Uncharacterized protein C9orf40 | | |
| 2 | 1 | sp\|O14810\|CPLX1_HUMAN Complexin-1 | 0.78 | 0.0195 |
| 2 | 1 | sp\|Q6PUV4\|CPLX2_HUMAN | 0.78 | 0.0195 |
| | | Complexin-2 | | |
| 2 | 1 | tr\|E5RG74\|E5RG74_HUMAN Brain- | 0.717 | 0.00332 |
| | | specific angiogenesis inhibitor 1 | | |
| 2 | 1 | tr\|E9PNW5\|E9PNW5_HUMAN | 0.745 | 0.0253 |
| | | Uncharacterized protein C4orf50 | | |
| 2 | 1 | tr\|I6Y0B1\|I6Y0B1_HUMAN MHC class | 0.795 | 0.0244 |
| | | I antigen (Fragment) | | |
| 2 | 1 | tr\|Q68D13\|Q68D13_HUMAN Putative | 0.765 | 0.0135 |
| | | uncharacterized protein DKFZp779C159 | | |
| | | (Fragment) | | |
| 2 | 1 | sp\|Q14160\|SCRIB_HUMAN Protein | 0.765 | 0.0124 |
| | | scribble homolog | | |
| 2 | 1 | sp\|Q6PGP7\|TTC37_HUMAN | 0.753 | 0.024599999 |
| | | Tetratricopeptide repeat protein 37 | | |
| 2 | 0 | sp\|Q16671\|AMHR2_HUMAN Anti- | 0.786 | 0.026699999 |
| | | Muellerian hormone type-2 receptor | | |
| 2 | 0 | tr\|B2RB52\|B2RB52_HUMAN cDNA, | 0.793 | 0.0383 |
| | | FLJ95314, highly similar to Homo sapiens | | |
| | | transducin (beta)-like 2 (TBL2), | | |
| | | transcript variant 1, mRNA | | |
| 2 | 0 | tr\|B2RBZ5\|B2RBZ5_HUMAN cDNA, | 0.796 | 0.044300001 |
| | | FLJ95778, highly similar to Homo sapiens | | |
| | | serpin peptidase inhibitor, clade A | | |
| | | (alpha-1 antiproteinase, antitrypsin), | | |
| | | member 10 (SERPINA10), mRNA | | |
| 2 | 0 | tr\|B4DG07\|B4DG07_HUMAN cDNA | 0.762 | 0.0484 |
| | | FLJ58159, highly similar to RAB6- | | |
| | | interacting protein 2 | | |
| 2 | 0 | tr\|G3V2W1\|G3V2W1_HUMAN Protein | 0.796 | 0.044300001 |
| | | Z-dependent protease inhibitor | | |
| 2 | 0 | tr\|H3BPI9\|H3BPI9_HUMAN Anti- | 0.786 | 0.026699999 |
| | | Muellerian hormone type-2 receptor | | |
| | | (Fragment) | | |
| 2 | 0 | sp\|Q9HDC5\|JPH1_HUMAN | 0.804 | 0.0308 |
| | | Junctophilin-1 | | |
| 2 | 0 | sp\|Q15784\|NDF2_HUMAN Neurogenic | 0.879 | 0.0449 |
| | | differentiation factor 2 | | |
| 2 | 0 | tr\|Q4KMX3\|Q4KMX3_HUMAN JPH1 | 0.804 | 0.0308 |
| | | protein (Fragment) | | |
| 2 | 0 | tr\|Q5U0R0\|Q5U0R0_HUMAN | 0.879 | 0.0449 |
| | | Neurogenic differentiation factor | | |
| 2 | 0 | tr\|Q7Z682\|Q7Z682_HUMAN Putative | 0.804 | 0.0308 |
| | | uncharacterized protein DKFZp779I2251 | | |
| | | (Fragment) | | |
| 2 | 0 | tr\|Q86VR1\|Q86VR1_HUMAN JPH1 | 0.804 | 0.0308 |
| | | protein (Fragment) | | |
| 2 | 0 | sp\|Q9UK55\|ZPI_HUMAN Protein Z- | 0.796 | 0.044300001 |
| | | dependent protease inhibitor | | |
| 3 | 1 | sp\|Q53TS8\|AL2SA_HUMAN | 0.795 | 0.0634 |
| | | Amyotrophic lateral sclerosis 2 | | |
| | | chromosomal region candidate gene 11 | | |
| | | protein | | |
| 3 | 1 | sp\|P01762\|HV301_HUMAN Ig heavy | 1.18 | 0.074199997 |
| | | chain V-III region TRO | | |
| 3 | 1 | sp\|A0M8Q6\|LAC7_HUMAN Ig lambda- | 1.13 | 0.078100003 |
| | | 7 chain C region | | |
| 3 | 1 | tr\|Q9UL88\|Q9UL88_HUMAN Myosin- | 1.18 | 0.074199997 |
| | | reactive immunoglobulin heavy chain | | |
| | | variable region (Fragment) | | |
| 3 | 0 | sp\|P52209\|6PGD_HUMAN 6- | 0.933 | 0.034600001 |
| | | phosphogluconate dehydrogenase, | | |
| | | decarboxylating | | |
| 3 | 0 | tr\|A0A075B6I8\|A0A075B6I8_HUMAN | 1.18 | 0.0451 |
| | | Protein IGLV1-47 (Fragment) | | |
| 3 | 0 | tr\|A0A075B6J8\|A0A075B6J8_HUMAN | 0.842 | 0.070600003 |
| | | Protein IGLV3-19 (Fragment) | | |
| 3 | 0 | tr\|A0PJD1\|A0PJD1_HUMAN ZNF200 | 1.19 | 0.0458 |
| | | protein (Fragment) | | |
| 3 | 0 | tr\|A2MYD0\|A2MYD0_HUMAN V1-17 | 1.18 | 0.0451 |
| | | protein (Fragment) | | |
| 3 | 0 | tr\|A4F255\|A4F255_HUMAN | 1.09 | 0.093000002 |
| | | Immunoblobulin G1 Fab heavy chain | | |
| | | variable region (Fragment) | | |
| 3 | 0 | tr\|B2R815\|B2R815_HUMAN cDNA, | 0.899 | 0.070799999 |
| | | FLJ93695, highly similar to Homo sapiens | | |
| | | serpin peptidase inhibitor, clade A | | |
| | | (alpha-1 antiproteinase, antitrypsin), | | |
| | | member 4 (SERPINA4), mRNA | | |
| 3 | 0 | tr\|B2R950\|B2R950_HUMAN cDNA, | 0.853 | 0.0506 |
| | | FLJ94213, highly similar to Homo sapiens | | |
| | | pregnancy-zone protein (PZP), mRNA | | |
| 3 | 0 | tr\|B3KNF3\|B3KNF3_HUMAN cDNA | 1.18 | 0.0265 |
| | | FLJ14501 fis, clone NT2RM1000199, | | |
| | | highly similar to Homo sapiens seizure | | |
| | | related 6 homolog-like 2 (SEZ6L2), | | |
| | | transcript variant 2, mRNA | | |
| 3 | 0 | tr\|B3KP91\|B3KP91_HUMAN cDNA | 1.19 | 0.0458 |
| | | FLJ31448 fis, clone NT2NE2000950, | | |
| | | highly similar to Zinc finger protein 200 | | |
| 3 | 0 | tr\|B7Z7M2\|B7Z7M2_HUMAN cDNA | 0.847 | 0.060899999 |
| | | FLJ51564, highly similar to Pregnancy | | |
| | | zone protein | | |
| 3 | 0 | tr\|B7ZMN7\|B7ZMN7_HUMAN LYST | 0.859 | 0.077699997 |
| | | protein | | |
| 3 | 0 | sp\|Q5M775\|CYTSB_HUMAN Cytospin-B | 1.17 | 0.046399999 |
| 3 | 0 | sp\|Q68D51\|DEN2C_HUMAN DENN | 0.793 | 0.057999998 |
| | | domain-containing protein 2C | | |
| 3 | 0 | tr\|E9KL26\|E9KL26_HUMAN | 0.901 | 0.090400003 |
| | | Epididymis tissue protein Li 173 | | |
| 3 | 0 | tr\|F8VY04\|F8VY04_HUMAN Adenylate | 0.909 | 0.059999999 |
| | | kinase 2, mitochondrial | | |
| 3 | 0 | tr\|F8VZG5\|F8VZG5_HUMAN Adenylate | 0.909 | 0.059999999 |
| | | kinase 2, mitochondrial | | |
| 3 | 0 | tr\|F8W1A4\|F8W1A4_HUMAN | 0.909 | 0.059999999 |
| | | Adenylate kinase 2, mitochondrial | | |
| 3 | 0 | tr\|F8W7L3\|F8W7L3_HUMAN Alpha-2- | 0.84 | 0.066600002 |
| | | macroglobulin (Fragment) | | |
| 3 | 0 | tr\|G3V213\|G3V213_HUMAN Adenylate | 0.909 | 0.059999999 |
| | | kinase 2, isoform CRA_a | | |
| 3 | 0 | tr\|H0YFH1\|H0YFH1_HUMAN Alpha-2- | 0.859 | 0.063500002 |
| | | macroglobulin (Fragment) | | |
| 3 | 0 | tr\|H0YJW9\|H0YJW9_HUMAN | 1.18 | 0.0506 |
| | | Uncharacterized protein (Fragment) | | |
| 3 | 0 | tr\|H3BN26\|H3BN26_HUMAN Seizure 6- | 1.18 | 0.0265 |
| | | like protein 2 (Fragment) | | |
| 3 | 0 | tr\|I3L1E4\|I3L1E4_HUMAN Zinc finger | 1.19 | 0.0458 |
| | | protein 200 (Fragment) | | |
| 3 | 0 | sp\|P05155\|IC1_HUMAN Plasma protease | 0.901 | 0.090400003 |
| | | C1 inhibitor | | |
| 3 | 0 | tr\|J7HH10\|J7HH10_HUMAN Vitronectin | 1.16 | 0.069499999 |
| | | (Fragment) | | |
| 3 | 0 | tr\|K7EM49\|K7EM49_HUMAN 6- | 0.933 | 0.034600001 |
| | | phosphogluconate dehydrogenase, | | |
| | | decarboxylating (Fragment) | | |
| 3 | 0 | tr\|K7EMN2\|K7EMN2_HUMAN 6- | 0.933 | 0.034600001 |
| | | phosphogluconate dehydrogenase, | | |
| | | decarboxylating (Fragment) | | |
| 3 | 0 | tr\|K7EPF6\|K7EPF6_HUMAN 6- | 0.933 | 0.034600001 |
| | | phosphogluconate dehydrogenase, | | |
| | | decarboxylating (Fragment) | | |
| 3 | 0 | sp\|P54819\|KAD2_HUMAN Adenylate | 0.909 | 0.059999999 |
| | | kinase 2, mitochondrial | | |
| 3 | 0 | sp\|P29622\|KAIN_HUMAN Kallistatin | 0.899 | 0.070799999 |
| 3 | 0 | sp\|P55268\|LAMB 2_HUMAN Laminin | 0.823 | 0.066299997 |
| | | subunit beta-2 | | |
| 3 | 0 | sp\|P01700\|LV102_HUMAN Ig lambda | 1.18 | 0.0451 |
| | | chain V-I region HA | | |
| 3 | 0 | sp\|P04208\|LV106_HUMAN Ig lambda | 1.18 | 0.0451 |
| | | chain V-I region WAH | | |
| 3 | 0 | sp\|Q99698\|LYST_HUMAN Lysosomal- | 0.859 | 0.077699997 |
| | | trafficking regulator | | |
| 3 | 0 | sp\|Q13219\|PAPP1_HUMAN Pappalysin-1 | 0.814 | 0.054099999 |
| 3 | 0 | sp\|P36955\|PEDF_HUMAN Pigment | 1.14 | 0.066600002 |
| | | epithelium-derived factor | | |
| 3 | 0 | sp\|Q92954\|PRG4_HUMAN Proteoglycan 4 | 1.19 | 0.064499997 |
| 3 | 0 | sp\|P20742\|PZP_HUMAN Pregnancy zone | 0.853 | 0.0506 |
| | | protein | | |
| 3 | 0 | tr\|Q5NV73\|Q5NV73_HUMAN V2-13 | 0.842 | 0.070600003 |
| | | protein (Fragment) | | |
| 3 | 0 | tr\|Q8N2F8\|Q8N2F8_HUMAN cDNA | 0.838 | 0.079499997 |
| | | PSEC0195 fis, clone HEMBA1001322, | | |
| | | highly similar to ALPHA-ADAPTIN C | | |
| 3 | 0 | tr\|Q9Y6X7\|Q9Y6X7_HUMAN | 0.855 | 0.0713 |
| | | KIAA0864 protein (Fragment) | | |
| 3 | 0 | sp\|Q9P2N5\|RBM27_HUMAN RNA- | 0.823 | 0.067900002 |
| | | binding protein 27 | | |
| 3 | 0 | sp\|Q6UXD5\|SE6L2_HUMAN Seizure 6- | 1.18 | 0.0265 |
| | | like protein 2 | | |
| 3 | 0 | sp\|Q9NUV7\|SPTC3_HUMAN Serine | 0.862 | 0.0682 |
| | | palmitoyltransferase 3 | | |
| 3 | 0 | sp\|P78524\|ST5_HUMAN Suppression of | 0.794 | 0.057 |
| | | tumorigenicity 5 protein | | |
| 3 | 0 | sp\|Q9ULT0\|TTC7A_HUMAN | 0.794 | 0.057 |
| | | Tetratricopeptide repeat protein 7A | | |
| 3 | 0 | tr\|U3KPZ7\|U3KPZ7_HUMAN RNA- | 0.823 | 0.067900002 |
| | | binding protein 27 | | |
| 3 | 0 | tr\|X5D2T7\|X5D2T7_HUMAN Seizure | 1.18 | 0.0265 |
| | | related 6-like protein 2 isoform E | | |
| | | (Fragment) | | |
| 3 | 0 | tr\|X5D7P3\|X5D7P3_HUMAN Seizure | 1.18 | 0.0265 |
| | | related 6-like protein 2 isoform B | | |
| | | (Fragment) | | |
| 3 | 0 | tr\|X5D9C2\|X5D9C2_HUMAN Seizure | 1.18 | 0.0265 |
| | | related 6-like protein 2 isoform G | | |
| | | (Fragment) | | |
| 3 | 0 | tr\|X5D9G4\|X5D9G4_HUMAN Seizure | 1.18 | 0.0265 |
| | | related 6-like protein 2 isoform C | | |
| 3 | 0 | tr\|X5DNZ5\|X5DNZ5_HUMAN Seizure | 1.18 | 0.0265 |
| | | related 6-like protein 2 isoform D | | |
| | | (Fragment) | | |
| 3 | 0 | sp\|P98182\|ZN200_HUMAN Zinc finger | 1.19 | 0.0458 |
| | | protein 200 | | |

Associated biological functions are noted in Table 2.

TABLE 2
Biological functions associated with differentially expressed
circulating exosomes/microparticles in 10-12 weeks gestation.

| GO Name | q-value |
|---|---|
| Negative Regulation of Epidermal Growth Factor Signaling | 1.07E−02 |
| Negative Regulation of Protein Dephosphorization | 1.60E−02 |
| Thrombin Receptor Signaling | 1.92E−02 |
| Cellular Hyperosmotic Response | 2.10E−02 |
| Cell Morphogenesis | 2.60E−02 |
| Negative Regulation of Necrotic Cell Death | 2.80E−02 |
| Glucocorticoid Signaling Pathway | 3.20E−02 |
| Regulation of DNA Dependent Transcription | 3.50E−02 |
| Protein Heterooligomerization | 3.60E−02 |
| Anatomical Structure Formation Involved in Morphogenesis | 3.81E−02 |
| Regulation of Sodium Ion Transmembrane Transporter | 4.76E−02 |
| Activity | |
| Regulation of Coagulation | 4.80E−02 |
| Stem Cell Differentiation | 4.20E−02 |
| Regulation of Complement Activity | 5.28E−02 |

Discussion: This study identifies a candidate set of CMP associated protein biomarkers at 10-12 weeks that demonstrate differential expression in pregnancies that go on to present with PE. Known protein functions indicate biological plausibility involving a variety of novel processes.

The protein biomarkers identified may be involved with key physiological and developmental processes, such as inter-related, systemic biological networks linked to coagulation, immune modulation, and the complement system, or localized tissue and cellular processes, such as cell death/differentiation, morphogenesis. Heretofore unknown processes or relationships between these processes, known or unknown to be involved in preeclampsia, may be identified. The functioning of these essential processes may be mediated, in part, by CMP interactions between various cells and tissues. The potential biological and clinical significance of this approach is in the non-invasive detection and monitoring of protein dysregulation in preeclampsias and possibly other obstetrical syndromes and conditions. Additionally, classifier models derived from protein biomarker quantification levels (microparticle-based tests) may be utilized to stratify risk of PE and treat at risk group with various interventions, including therapeutic.

### Example 3: Biomarkers and Biomarker Panels for Risk of Preeclampsia.

A pipeline was created for supervised CMP-associated protein classification. The list of identified peptides and proteins was submitted to the STRING database for known protein interactions. string-db.org/. Those proteins with greater than 5 documented interactions were retained. Block randomization was used to divide the data into training and test sets. Within the training set, ensemble feature selection was used to create a subset of the most informative individual proteins that were significantly and consistently associated with preeclampsia versus controls. 5-fold cross validation using logistic regression modeling was then used to examine the information content of all possible multivariate models drawn from this subset. The best performing cross validated candidate models were then run against the test set to establish performance on independent data. Protein function was determined with reference to the UniProt database.

Machine learning methods used to generate predictive models involved several aspects “ensemble feature selection”, “logistic regression”, and “permutation analysis”.

The molecular function of the top candidate CMP-associated proteins were associated with various important cellular and blood-based biological functions including coagulation and platelet activation, cell adhesion (cell-to-cell and cell-to matrix), migration and chemotaxis, cell proliferation, cellular differentiation and morphogenesis, angiogenesis, adipocyte lipid metabolism, lipoprotein metabolism, lipoprotein lipase activity, cholesterol biosynthesis, intracellular organization of sub-cellular structures (especially for the sarcoplasmic and endoplasmic reticulum), calcium release and signaling, complement activation and membrane attack complex assembly, the innate immune response, endopeptidase inhibition, microtubular-based ciliary movement and sperm motility, ER stress, and neurotransmitter and neuropeptide exocytosis.

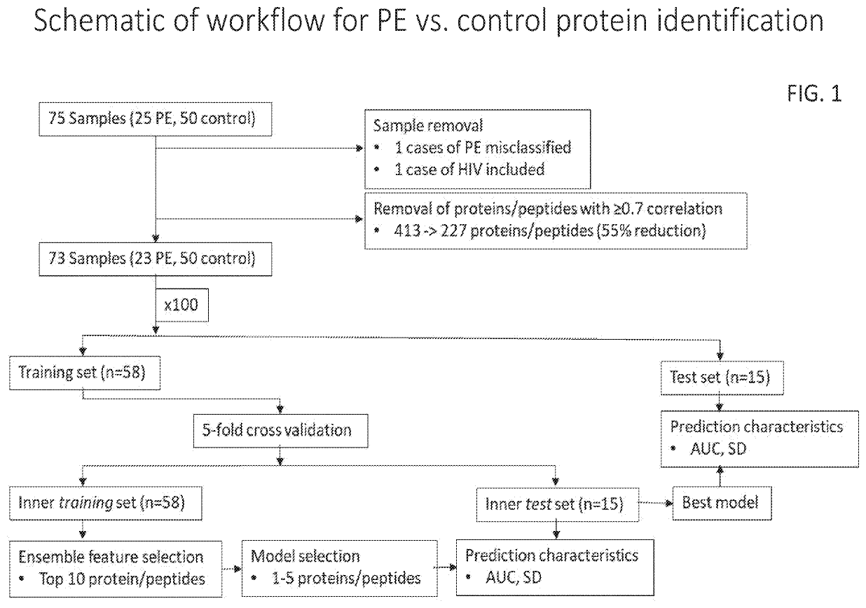

FIG. 1 shows a schematic workflow for identifying biomarkers and panels of biomarkers for risk of preeclampsia. The workflow includes the following operations: Samples for studies are provided. In this case, of 75 original samples, 73 were selected for study, 23 of which were from preeclampsia subjects and 50 of which were controls. The samples were divided into a training set of 58 samples and a test set of 15 samples.

### Initial Machine Learning Analysis

Quantitative measures of proteins in each of the samples in the training set were determined. These measures were analyzed by machine learning to develop models to predict risk of preeclampsia. The highest performing models that included panels of 3 to 5 protein biomarkers were selected. Five-fold cross validation was used. Performance was a function of area under the curve (AUC). The best performing models from this first round of internal testing are presented in FIG. 3. Proteins are identified by protein name, gene name or accession number in any of a variety of publicly available protein databases such as, for example, SwissProt.

Further identifying information for certain of these proteins is set forth in Table 3.

TABLE 3

| Listed | Protein names | Gene names | Length |
|---|---|---|---|
| A2N0U6_HUMAN | VH6DJ protein (Fragment) | VH6DJ | 116 |
| A0A024R8D8_HUMAN | Progestagen-associated | PAEP | 180 |
| | endometrial protein (Placental | hCG_28728 | |
| | protein 14, pregnancy- | | |
| | associated endometrial alpha- | | |
| | 2-globulin, alpha uterine | | |
| | protein), isoform CRA_d | | |
| B2R6L0_HUMAN | Tubulin beta chain | | 445 |
| GP1BA_HUMAN | Platelet glycoprotein Ib alpha | GP1BA | 652 |
| | chain (GP-Ib alpha) (GPIb- | | |
| | alpha) (GPIbA) (Glycoprotein | | |
| | Ibalpha) (Antigen CD42b- | | |
| | alpha) (CD antigen CD42b) | | |
| | [Cleaved into: Glycocalicin] | | |
| Q96TB4_HUMAN | Envelope protein (Fragment) | env | 180 |
| Q5NV82_HUMAN | V4-2 protein (Fragment) | V4-2 | 104 |
| E3UVQ2_HUMAN | BCL6 corepressor/retinoic | BCOR-RARA | 1931 |
| | acid receptor alpha fusion | | |
| | protein | | |
| E9PQG4_HUMAN | Myomegalin | PDE4DIP | 740 |
| L0R6N9_HUMAN | Alternative protein SETD1A | SETD1A | 340 |
| VTNC_HUMAN | Vitronectin (VN) (S-protein) | VTN | 478 |
| | (Serum-spreading factor) | | |
| | (V75) [Cleaved into: | | |
| | Vitronectin V65 subunit; | | |
| | Vitronectin V10 subunit; | | |
| | Somatomedin-B] | | |
| C1RL_HUMAN | Complement C1r | C1RL C1RL1 | 487 |
| | subcomponent-like protein | C1RLP CLSPA | |
| | (C1r-LP) (C1r-like protein) | | |
| | (EC 3.4.21.-) (C1r-like serine | | |
| | protease analog protein) | | |
| | (CLSPa) | | |
| MBL2_HUMAN | Mannose-binding protein C | MBL2 COLEC1 | 248 |
| | (MBP-C) (Collectin-1) | MBL | |
| | (MBP1) (Mannan-binding | | |
| | protein) (Mannose-binding | | |
| | lectin) | | |
| B2R815_HUMAN | cDNA, FLJ93695, highly | | 427 |
| | similar to Homo sapiens | | |
| | serpin peptidase inhibitor, | | |
| | clade A (alpha-1 | | |
| | antiproteinase, antitrypsin), | | |
| | member 4 (SERPINA4), | | |
| | mRNA | | |
| D6MJD1_HUMAN | MHC class I antigen | HLA-A | 181 |
| | (Fragment) | | |
| ZA2G_HUMAN | Zinc-alpha-2-glycoprotein | AZGP1 ZAG | 298 |
| | (Zn-alpha-2-GP) (Zn-alpha-2- | ZNGP1 | |
| | glycoprotein) | | |
| A0A024R9I2_HUMAN | Muscarinic acetylcholine | CHRM5 | 532 |
| | receptor | hCG_37416 | |
| TPC11_HUMAN | Trafficking protein particle | TRAPPC11 | 1133 |
| | complex subunit 11 | C4orf41 | |
| CO5_HUMAN | Complement C5 (C3 and | C5 CPAMD4 | 1676 |
| | PZP-like alpha-2- | | |
| | macroglobulin domain- | | |
| | containing protein 4) [Cleaved | | |
| | into: Complement C5 beta | | |
| | chain; Complement C5 alpha | | |
| | chain; C5a anaphylatoxin; | | |
| | Complement C5 alpha′ chain] | | |
| A0A024R3Z1_HUMAN | Microtubule-associated | MAP2 | 1858 |
| | protein | hCG_1776452 | |
| A8K008_HUMAN | Uncharacterized protein | | 472 |
| B2R4C5_HUMAN | Lysozyme (EC 3.2.1.17) | LYZ LYZF1 | 148 |
| | | hCG_24462 | |
| B4E1D8_HUMAN | cDNA FLJ51597, highly | | 536 |
| | similar to C4b-binding protein | | |
| | alpha chain | | |
| GP112_HUMAN | Adhesion G-protein coupled | ADGRG4 | 3080 |
| | receptor G4 (G-protein | GPR112 | |
| | coupled receptor 112) | | |
| F8VY04_HUMAN | Adenylate kinase 2, | AK2 | 190 |
| | mitochondrial | | |
| AACT_HUMAN | Alpha-1-antichymotrypsin | SERPINA3 | 423 |
| | (ACT) (Cell growth-inhibiting | AACT GIG24 | |
| | gene 24/25 protein) (Serpin | GIG25 | |
| | A3) [Cleaved into: Alpha-1- | | |
| | antichymotrypsin His-Pro- | | |
| | less] | | |
| B7ZKK7_HUMAN | eIF2AK2 protein | EIF2AK2 | 546 |
| FA11_HUMAN | Coagulation factor XI (FXI) | F11 | 625 |
| | (EC 3.4.21.27) (Plasma | | |
| | thromboplastin antecedent) | | |
| | (PTA) [Cleaved into: | | |
| | Coagulation factor XIa heavy | | |
| | chain; Coagulation factor XIa | | |
| | light chain] | | |
| M0QZN2_HUMAN | 40S ribosomal protein S5 | RPS5 | 134 |
| A0A024RAW9_HUMAN | WW domain binding protein | WBP11 | 641 |
| | 11, isoform CRA_a | hCG_24415 | |
| A2MYE2_HUMAN | A30 protein (Fragment) | A30 | 96 |
| APOC2_HUMAN | Apolipoprotein C-II (Apo- | APOC2 APC2 | 101 |
| | CII) (ApoC-II) | | |
| | (Apolipoprotein C2) [Cleaved | | |
| | into: Proapolipoprotein C-II | | |
| | (ProapoC-II)] | | |
| APOD_HUMAN | Apolipoprotein D (Apo-D) | APOD | 189 |
| | (ApoD) | | |
| APOH_HUMAN | Beta-2-glycoprotein 1 (APC | APOH B2G1 | 345 |
| | inhibitor) (Activated protein | | |
| | C-binding protein) | | |
| | (Anticardiolipin cofactor) | | |
| | (Apolipoprotein H) (Apo-H) | | |
| | (Beta-2-glycoprotein I) | | |
| | (B2GPI) (Beta(2)GPI) | | |
| B4DDG3_HUMAN | cDNA FLJ51688, highly | | 418 |
| | similar to Cleavage | | |
| | stimulation factor 50 kDa | | |
| | subunit | | |
| CAPS1_HUMAN | Calcium-dependent secretion | CADPS CAPS | 1353 |
| | activator 1 (Calcium- | CAPS1 | |
| | dependent activator protein | KIAA1121 | |
| | for secretion 1) (CAPS-1) | | |
| DYH3_HUMAN | Dynein heavy chain 3, | DNAH3 | 4116 |
| | axonemal (Axonemal beta | DNAHC3B | |
| | dynein heavy chain 3) | | |
| | (HsADHC3) (Ciliary dynein | | |
| | heavy chain 3) (Dnahc3-b) | | |
| E7EVP7_HUMAN | Deleted. | | |
| F8VV57_HUMAN | Keratin, type II cytoskeletal 5 | KRT5 | 132 |
| | (Fragment) | | |
| HEP2_HUMAN | Heparin cofactor 2 (Heparin | SERPIND1 | 499 |
| | cofactor II) (HC-II) (Protease | HCF2 | |
| | inhibitor leuserpin-2) (HLS2) | | |
| | (Serpin D1) | | |
| JPH1_HUMAN | Junctophilin-1 (JP-1) | JPH1 JP1 | 661 |
| | (Junctophilin type 1) | | |
| LCAT_HUMAN | Phosphatidylcholine-sterol | LCAT | 440 |
| | acyltransferase (EC 2.3.1.43) | | |
| | (Lecithin-cholesterol | | |
| | acyltransferase) | | |
| | (Phospholipid-cholesterol | | |
| | acyltransferase) | | |
| PIGR_HUMAN | Polymeric immunoglobulin | PIGR | 764 |
| | receptor (PIgR) (Poly-Ig | | |
| | receptor) (Hepatocellular | | |
| | carcinoma-associated protein | | |
| | TB6) [Cleaved into: Secretory | | |
| | component] | | |
| Q59EP2_HUMAN | Angiotensinogen variant | | 491 |
| | (Fragment) | | |
| Q5NV90_HUMAN | V2-17 protein (Fragment) | V2-17 | 97 |
| Q8IWX2_HUMAN | Hyaluronan binding protein | HABP2 | 516 |
| | (Fragment) | | |
| TSP1_HUMAN | Thrombospondin-1 | THBS1 TSP | 1170 |
| | (Glycoprotein G) | TSP1 | |

The resulting models were then validated against data from the test set of samples. The best performing models from this validation step are presented in FIG. 4A and FIG. 4B.

The frequency of occurrence of proteins in the highest performing models at this validation stop is presented below, in Table 4.

TABLE 4

| | Protein | Frequency |
|---|---|---|
| 1 | A2N0U6 | 87 |
| 2 | A0A024R8D8 | 68 |
| 3 | B2R6L0 | 27 |
| 4 | GP1BA | 25 |
| 5 | Q96TB4 | 24 |
| 6 | A0A075B6I4 | 19 |
| 7 | Q5NV82 | 19 |
| 8 | E3UVQ2 | 13 |
| 9 | E9PQG4 | 12 |
| 10 | L0R6N9 | 12 |
| 11 | VTNC | 12 |
| 12 | C1RL | 11 |
| 13 | MBL2 | 10 |
| 14 | B2R815 | 9 |
| 15 | D6MJD1 | 9 |
| 16 | ZA2G | 9 |
| 17 | A0A024R9I2 | 7 |
| 18 | TPC11 | 7 |
| 19 | CO5 | 6 |
| 20 | A0A024R3Z1 | 5 |
| 21 | A8K008 | 4 |
| 22 | B2R4C5 | 4 |
| 23 | B4E1D8 | 4 |
| 24 | GP112 | 4 |
| 25 | A0A075B6H9 | 3 |

Next, proteins identified in the previous model building step were compared against the STRING protein database. string-db.org/. Proteins that were (i) present in that database and (ii) networked with at least four of the proteins in the database, were selected for further study. (FIG. 1—“removal of proteins/peptides without annotation in STRING database and >4 edges in network.)

New models using the selected proteins were generated and biomarker panels with the highest performance as measured by area under the curve were selected. These models are presented in FIG. 5. (Panels 1-24.)

Table 5, below, provides protein biomarkers for preeclampsia and the frequency with which these biomarkers appeared in biomarker panels generated by machine learning.

TABLE 5

| | Protein | Frequency |
|---|---|---|
| 1 | GP1BA | 79 |
| 2 | VTNC | 57 |
| 3 | C1RL | 49 |
| 4 | ZA2G | 46 |
| 5 | APOC2 | 37 |
| 6 | APOH | 30 |
| 7 | JPH1 | 28 |
| 8 | CO5 | 16 |
| 9 | HEP2 | 16 |
| 10 | TPC11 | 14 |
| 11 | MBL2 | 11 |
| 12 | AACT | 8 |
| 13 | DYH3 | 7 |
| 14 | TSP1 | 7 |
| 15 | CAPS1 | 6 |
| 16 | APOD | 3 |
| 17 | LCAT | 1 |

Table 6, below, provides information about protein biomarkers set forth in Table 5.

TABLE 6

| Protein | Protein names | Gene names | Length |
|---|---|---|---|
| GP1BA_HUMAN | Platelet glycoprotein Ib alpha | GP1BA | 652 |
| | chain (GP-Ib alpha) (GPIb- | | |
| | alpha) (GPIbA) (Glycoprotein | | |
| | Ibalpha) (Antigen CD42b- | | |
| | alpha) (CD antigen CD42b) | | |
| | [Cleaved into: Glycocalicin] | | |
| VTNC_HUMAN | Vitronectin (VN) (S-protein) | VTN | 478 |
| | (Serum-spreading factor) | | |
| | (V75) [Cleaved into: | | |
| | Vitronectin V65 subunit; | | |
| | Vitronectin V10 subunit; | | |
| | Somatomedin-B] | | |
| C1RL_HUMAN | Complement C1r | C1RL C1RL1 | 487 |
| | subcomponent-like protein | C1RLP CLSPA | |
| | (C1r-LP) (C1r-like protein) | | |
| | (EC 3.4.21.-) (C1r-like serine | | |
| | protease analog protein) | | |
| | (CLSPa) | | |
| ZA2G_HUMAN | Zinc-alpha-2-glycoprotein | AZGP1 ZAG | 298 |
| | (Zn-alpha-2-GP) (Zn-alpha-2- | ZNGP1 | |
| | glycoprotein) | | |
| APOC2_HUMAN | Apolipoprotein C-II (Apo- | APOC2 APC2 | 101 |
| | CII) (ApoC-II) | | |
| | (Apolipoprotein C2) [Cleaved | | |
| | into: Proapolipoprotein C-II | | |
| | (ProapoC-II)] | | |
| APOH_HUMAN | Beta-2-glycoprotein 1 (APC | APOH B2G1 | 345 |
| | inhibitor) (Activated protein | | |
| | C-binding protein) | | |
| | (Anticardiolipin cofactor) | | |
| | (Apolipoprotein H) (Apo-H) | | |
| | (Beta-2-glycoprotein I) | | |
| | (B2GPI) (Beta(2)GPI) | | |
| JPH1_HUMAN | Junctophilin-1 (JP-1) | JPH1 JP1 | 661 |
| | (Junctophilin type 1) | | |
| CO5_HUMAN | Complement C5 (C3 and | C5 CPAMD4 | 1676 |
| | PZP-like alpha-2- | | |
| | macroglobulin domain- | | |
| | containing protein 4) [Cleaved | | |
| | into: Complement C5 beta | | |
| | chain; Complement C5 alpha | | |
| | chain; C5a anaphylatoxin; | | |
| | Complement C5 alpha′ chain] | | |
| HEP2_HUMAN | Heparin cofactor 2 (Heparin | SERPIND1 | 499 |
| | cofactor II) (HC-II) (Protease | HCF2 | |
| | inhibitor leuserpin-2) (HLS2) | | |
| | (Serpin D1) | | |
| TPC11_HUMAN | Trafficking protein particle | TRAPPC11 | 1133 |
| | complex subunit 11 | C4orf41 | |
| MBL2_HUMAN | Mannose-binding protein C | MBL2 COLEC1 | 248 |
| | (MBP-C) (Collectin-1) | MBL | |
| | (MBP1) (Mannan-binding | | |
| | protein) (Mannose-binding | | |
| | lectin) | | |
| AACT_HUMAN | Alpha-1-antichymotrypsin | SERPINA3 | 423 |
| | (ACT) (Cell growth-inhibiting | AACT GIG24 | |
| | gene 24/25 protein) (Serpin | GIG25 | |
| | A3) [Cleaved into: Alpha-1- | | |
| | antichymotrypsin His-Pro- | | |
| | less] | | |
| DYH3_HUMAN | Dynein heavy chain 3, | DNAH3 | 4116 |
| | axonemal (Axonemal beta | DNAHC3B | |
| | dynein heavy chain 3) | | |
| | (HsADHC3) (Ciliary dynein | | |
| | heavy chain 3) (Dnahc3-b) | | |
| TSP1_HUMAN | Thrombospondin-1 | THBS1 TSP | 1170 |
| | (Glycoprotein G) | TSP1 | |
| CAPS1_HUMAN | Calcium-dependent secretion | CADPS CAPS | 1353 |
| | activator 1 (Calcium- | CAPS1 | |
| | dependent activator protein | KIAA1121 | |
| | for secretion 1) (CAPS-1) | | |
| APOD _HUMAN | Apolipoprotein D (Apo-D) | APOD | 189 |
| | (ApoD) | | |
| LCAT_HUMAN | Phosphatidylcholine-sterol | LCAT | 440 |
| | acyltransferase (EC 2.3.1.43) | | |
| | (Lecithin-cholesterol | | |
| | acyltransferase) | | |
| | (Phospholipid-cholesterol | | |
| | acyltransferase) | | |

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

What is claimed is:

1. A method for assessing risk of preeclampsia in a pregnant subject, the method comprising:
(a) preparing a microparticle-enriched fraction from a blood sample from the pregnant subject;
(b) determining a quantitative measure of one or more microparticle-associated protein biomarkers in the fraction, wherein the one or more protein biomarkers are selected from:
(i) a protein biomarker of Table 1;
(ii) a protein biomarker of the set: A2N0U6, A0A024R8D8, B2R6L0, GP1BA, Q96TB4, A0A075B6I4, Q5NV82, E3UVQ2, E9PQG4, L0R6N9, VTNC, C1RL, MBL2, B2R815, D6MJD1, ZA2G, A0A024R912, TPC11, CO5, A0A024R3Z1, A8K008, B2R4C5, B4E1D8, GP112, A0A075B6H9; and
(iii) a protein biomarker of the set: GP1BA, VTNC, C1RL, ZA2G, APOC2, APOH, JPH1, CO5, HEP2, TPC11, MBL2, AACT, DYH3, TSP1, CAPS1, APOD, LCAT; and
(c) assessing the risk of preeclampsia based on the measure.

2. The method of claim 1, wherein an increased amount of an up-regulated biomarker or a decreased amount of a down-regulated biomarker indicates increased risk of preeclampsia.

3. The method of claim 1, comprising determining a quantitative measure of a plurality of protein biomarkers selected from the protein biomarkers of Table 1.

4. The method of claim 1, wherein the one or more protein biomarkers are selected from Table 1: Group 1, Group 2 or Group 3.

5. The method of claim 1, wherein the one or more protein biomarkers are selected from each of a plurality of biological functions selected from immune function, cell signaling, angiogenesis, apoptosis, matrix attachment, cell function, protein metabolism, ion transport and unknown function.

6. The method of claim 1, comprising determining risk of severe preeclampsia wherein the biomarker or biomarkers are selected from: 0A075B6I5_HUMAN, A2MYD2_HUMAN, AL2SA_HUMAN, AR13B_HUMAN, B3AT_HUMAN, BAI1_HUMAN, BRWD3_HUMAN, C6K6H8_HUMAN, CI040_HUMAN, CPLX1_HUMAN, CPLX2_HUMAN, E5RG74_HUMAN, E9PNW5_HUMAN, HV301_HUMAN, I6Y0B1_HUMAN, J3KPJ3_HUMAN, LAC7_HUMAN, LIPA2_HUMAN, LV104_HUMAN, LV109_HUMAN, Q68D13_HUMAN, Q9UL88_HUMAN, SCRIB_HUMAN and TTC37_HUMAN.

7. The method of claim 1, comprising determining a quantitative measure of a plurality of protein biomarkers selected from A2N0U6, A0A024R8D8, B2R6L0, GP1BA, Q96TB4, A0A075B614, Q5NV82, E3UVQ2, E9PQG4, L0R6N9, VTNC, C1RL, MBL2, B2R815, D6MJD1, ZA2G, A0A024R912, TPC11, CO5, A0A024R3Z1, A8K008, B2R4C5, B4E1D8, GP112, and A0A075B6H9.

8. The method of claim 1, comprising determining a quantitative measure of a plurality of protein biomarkers selected from GP1BA, VTNC, C1RL, ZA2G, APOC2, APOH, JPH1, CO5, HEP2, TPC11, MBL2, AACT, DYH3, TSP1, CAPS1, APOD, and LCAT.

9. The method of claim 1, wherein the biomarkers comprise a panel of biomarkers selected from panels 1-29 (FIG. 3), panels 1-56 (FIGS. 4A-4B) and panels 1-24 (FIG. 5).

10. The method of claim 9, wherein the panel comprises no more than any of 10, 9, 8, 7, 6, 5, 4 or 3 protein biomarkers.

11. The method of claim 1, wherein the biomarkers consist of a panel of biomarkers selected from panels 1-29 (FIG. 3), panels 1-56 (FIGS. 4A-4B) and panels 1-24 (FIG. 5).

12. The method of claim 1, wherein the biomarkers comprise a panel of biomarkers including 5, 4, 3 or 2 biomarkers selected from A2N0U6, A0A024R8D8, B2R6L0, GP1BA and Q96TB4.

13. The method of claim 1, wherein the biomarkers comprise a panel of biomarkers including A2N0U6 and at least 1, 2, 3, or 4 of A0A024R8D8, B2R6L0, GP1BA and Q96TB4.

14. The method of claim 1, wherein the biomarkers comprise a panel of biomarkers including 6, 5, 4, 3 or 2 biomarkers selected from GP1BA, VTNC, C1RL, ZA2G, APOC2 and APOH.

15. The method of claim 1, wherein the biomarkers comprise a panel of biomarkers including GP1BA and at least 1, 2, 3, 4 or 5 of VTNC, C1RL, ZA2G, APOC2 and APOH.

16. The method of any one of the preceding claims, wherein the sample is taken from the pregnant subject during the first trimester or second trimester of pregnancy.

17. The method of claim 7, wherein the sample is taken from the pregnant subject during weeks 10-12 of gestation.

18. The method of any of the preceding claims, wherein the pregnant subject is primigravida, multigravida, primiparous or multiparous.

19. The method of any of the preceding claims, wherein the pregnant subject has a singleton pregnancy or multiple pregnancy.

20. The method of any of the preceding claims, wherein the pregnant subject is asymptomatic for preeclampsia, e.g., is not hypertensive or does not have proteinuria.

21. The method of any of the preceding claims, wherein the pregnant subject has no history of preeclampsia.

22. The method of any of the preceding claims, wherein the pregnant subject has no risk factors for preeclampsia.

23. The method of any of the preceding claims, wherein the pregnant subject has chronic hypertension.

24. The method of any of the preceding claims, wherein the blood sample is plasma or serum.

25. The method of any of the preceding claims, wherein the microparticle-enriched fraction is prepared using size-exclusion chromatography.

26. The method of claim 25, wherein the size-exclusion chromatography comprises elution with water.

27. The method of claim 25, wherein the size-exclusion chromatography is performed with an agarose solid phase and an aqueous liquid phase.

28. The method of claim 25, wherein the preparing step further comprises using ultrafiltration or reverse-phase chromatography.

29. The method of claim 25, wherein the preparing step further comprises denaturation using urea, reduction using dithiothreitol, alkylation using iodoacetamine, and digestion using trypsin after the size exclusion chromatography.

30. The method of claim 1, wherein the microparticles are further purified to enrich for placental-derived exosomes or vascular endothelial-derived exosomes.

31. The method of any of the preceding claims, wherein determining a quantitative measure comprises mass spectrometry.

32. The method of claim 31, wherein determining a quantitative measure comprises liquid chromatography/mass spectrometry (LC/MS).

33. The method of claim 31, wherein mass spectrometry comprises liquid chromatography/triple quadrupole mass spectrometry.

34. The method of claim 31, wherein the mass spectrometry comprises multiple reaction monitoring.

35. The method of claim 31, wherein the mass spectrometry comprises multiple reaction monitoring, and the liquid chromatography is done using a solvent comprising acetonitrile, and/or determining comprises assigning an indexed retention time to the protein biomarkers.

36. The method of claim 31, wherein the mass spectrometry comprises multiple reaction monitoring, and the method comprises adding one or more stable isotope standard peptides to the sample before introduction into the mass spectrometer and detection comprises detecting one or a plurality of daughter ions of the stable isotope peptide standards produced by a collision cell of the mass spectrometer.

37. The method of claim 31, wherein determining the quantitative measure comprises determining a quantitative measure of a surrogate peptide of the protein biomarker.

38. The method of claim 36, wherein mass spectrometry comprises quantifying one or more stable isotope labeled standard peptides (SIS peptides) corresponding to each of the surrogate peptides.

39. The method of claim 31, comprising adding one or more stable heavy isotope substituted standards corresponding to said protein biomarkers to the microparticle enriched fraction.

40. The method of any of claims 1-30, wherein determining a quantitative measure comprises contacting the sample with one or more capture reagents, each capture reagent specifically binding one of the protein biomarkers, and detecting binding between the capture reagent in the protein biomarker.

41. The method of claim 39, comprising performing an immunoassay.

42. The method of claim 40, wherein the immunoassay is selected from the group consisting of enzyme immunoassay (EIA), enzyme-linked immunosorbent assay (ELISA), and radioimmunoassay (RIA).

43. The method of any of the preceding claims, wherein the assessing comprises executing a classification rule, which rule classifies the subject at being at risk of preeclampsia, and wherein execution of the classification rule produces a correlation between preeclampsia or term birth with a p value of less than at least 0.05.

44. The method of any of the preceding claims, wherein the assessing comprises executing a classification rule, which rule classifies the subject at being at risk of preeclampsia, and wherein execution of the classification rule produces a receiver operating characteristic (ROC) curve, wherein the ROC curve has an area under the curve (AUC) of at least 0.6, at least 0.7, at least 0.8 or at least 0.9.

45. The method of any of the preceding claims, wherein values on which the classification rule classifies a subject further include at least one of: maternal age, maternal body mass index, primiparous, and smoking during pregnancy.

46. The method of any of the preceding claims, wherein the classification rule employs cut-off, linear regression (e.g., multiple linear regression (MLR), partial least squares (PLS) regression, principal components regression (PCR)), binary decision trees (e.g., recursive partitioning processes such as CART-classification and regression trees), artificial neural networks such as back propagation networks, discriminant analyses (e.g., Bayesian classifier or Fischer analysis), logistic classifiers, and support vector classifiers (e.g., support vector machines).

47. The method of any of the preceding claims, wherein the classification rule is configured to have a sensitivity, specificity, positive predictive value or negative predictive value of at least 70%, least 80%, at least 90% or at least 95%.

48. The method of any of the preceding claims, wherein assessing an increased risk of preeclampsia comprises determining that the protein biomarker (if upregulated) is above or (if down regulated) is below a threshold level.

49. The method of claim 47, wherein the threshold level represents a level at least one, at least two or at least three z scores from a measure of central tendency (e.g., mean, median or mode) for the protein determined from at least 50, at least 100 or at least 200 control subjects.

50. The method of any of the preceding claims, wherein the assessing comprises comparing the measure of each protein in the panel to a reference standard.

51. The method of any of the preceding claims, further comprising communicating the risk of preeclampsia for a pregnant subject to a health care provider.

52. The method of any of the preceding claims further comprising:
(d) determining, a quantitative measure of one or more microparticle-associated protein biomarkers for preterm birth in the fraction; and
(e) assessing the risk of preterm birth based on the measure.

53. A method of decreasing risk of preeclampsia for a pregnant subject and/or reducing neonatal complications of preeclampsia, the method comprising:
(a) assessing risk of preeclampsia for a pregnant subject according to the method of any one of claims 1 to 50; and
(b) administering a therapeutic intervention to the subject effective to decrease the risk of preeclampsia and/or reduce neonatal complications of preeclampsia.

54. The method of claim 52, wherein the therapeutic intervention is selected from the group consisting of aspirin (e.g., low dose aspirin), a corticosteroid or a medication to reduce hypertension.

55. The method of claim 52, wherein the preeclampsia treated is a later or milder form, hypertensive form or earlier or severe form.

56. A method comprising administering to a pregnant subject determined to have an increased risk of preeclampsia by a method as described herein, a therapeutic intervention effective to reduce the risk of preeclampsia or to reduce neonatal complications of preeclampsia.

57. A method of administering to a pregnant subject having an altered quantitative measure as compared to a reference standard of any one of the panels of protein biomarkers selected from panels 1-29 (FIG. 3), panels 1-56 (FIGS. 4A-4B) and panels 1-24 (FIG. 5), an effective amount of a treatment designed to reduce the risk of preeclampsia.

58. A panel comprising a plurality of substantially pure protein biomarkers or surrogate biomarkers selected from the protein biomarkers of Table 1, Table 3 or Table 4.

59. The panel of claim 57 further comprising a stable isotope standard peptide paired with each of the surrogate biomarkers.

60. A kit comprising one or a plurality of containers, wherein each container comprises one or more of each of a plurality of Stable Isotopic Standards, each stable isotopic standard corresponding to a surrogate peptide for a biomarker from a panel of biomarkers selected from panels 1-29 (FIG. 3), panels 1-56 (FIGS. 4A-4B) and panels 1-24 (FIG. 5).

61. A computer readable medium in tangible, non-transitory form comprising code to implement a classification rule generated by a method as described herein.

62. A system comprising:
(a) a computer comprising:
(i) a processor; and
(II) a memory, coupled to the processor, the memory storing a module comprising:
(1) test data for a sample from a subject including values indicating a quantitative measure of one or more protein biomarkers in the fraction, wherein the protein biomarkers are selected from the protein biomarkers of Table 1, Table 3 and Table 4;
(2) a classification rule which, based on values including the measurements, classifies the subject as being at risk of pre-term birth, wherein the classification rule is configured to have a sensitivity of at least 75%, at least 85% or at least 95%; and
(3) computer executable instructions for implementing the classification rule on the test data.
